(19) United States
(12) Patent Application Publication
(10) Pub. No.: US 2025/0241528 A1
(43) Pub. Date: Jul. 31, 2025

(54) Laparoscopic Oximetry Probe and Method for Reuse of a Handheld Unit of the Laparoscopic Oximetry Probe

(71) Applicant: ViOptix, Inc., Newark, CA (US)

(72) Inventors: Alex Michael Margiott, Dunbarton, NH (US); Sean Gossin, Media, PA (US); Timothy Lee Sauder, San Francisco, CA (US); Kevin Dunk, Casto Valley, CA (US); Scott E. Coleridge, New York, NY (US); Mark Lonsinger, San Jose, CA (US)

(21) Appl. No.: 19/014,124

(22) Filed: Jan. 8, 2025

Related U.S. Application Data
(60) Provisional application No. 63/618,864, filed on Jan. 8, 2024.

(51) Int. Cl. A61B 1/313; A61B 5/1455

(52) U.S. Cl. CPC A61B 1/3132; A61B 5/1455

(57) ABSTRACT

A laparoscopic medical device includes an oximeter sensor at its tip, which allows making oxygen saturation measurements laparoscopically. The laparoscopic medical device includes a probe unit and a laparoscopic tube that detachably connects with the probe unit so that the laparoscopic tube can be replaced for different patient surgeries and the probe unit can be reused for the different surgeries. The probe unit includes a number of transmitting optical fibers and the detachable laparoscopic tube includes a number of receiving optical fibers where tips of the transmitting and receiving optical fibers connect end to end. Cores of the transmitting optical fibers have smaller numerical apertures than cores of the receiving optical fibers to facilitate a high percentage of light transmission from the transmitting optical fibers to the receiving optical fibers even when the cores of the transmitting and receiving optical fibers are misaligned at their connecting ends.

### CROSS-REFERENCE TO RELATED APPLICATIONS

This application claims the benefit of U.S. patent application 63/618,864, filed Jan. 8, 2024, which is incorporated by reference along with all other references cited in this application.

### BACKGROUND OF THE INVENTION

The invention generally relates to optical systems that monitor oxygen levels in tissue. More specifically, the invention relates to an optical probe, such as a laparoscopic oximeter, that includes a handheld probe unit that is reusable and a laparoscopic tube that is detachable from the probe unit, is disposable after use, and where another laparoscopic tube may replace the laparoscopic tube that has been disposed of.

Oximeters are medical devices used to measure the oxygen saturation of tissue in humans and living things for various purposes. For example, oximeters are used for medical and diagnostic purposes in hospitals and other medical facilities (e.g., surgery, patient monitoring, or ambulance or other mobile monitoring for, e.g., hypoxia); sports and athletics purposes at a sports arena (e.g., professional athlete monitoring); personal or at-home monitoring of individuals (e.g., general health monitoring, or personal training for a marathon); and veterinary purposes (e.g., animal monitoring).

Pulse oximeters and tissue oximeters are two types of oximeters that operate on different principles. A pulse oximeter uses a patient's pulse to make measurements. A pulse oximeter typically measures the absorbance of light due to pulsing arterial blood. In contrast, a tissue oximeter does not need a pulse in order to function and can be used to make oxygen saturation measurements of a tissue flap that has been disconnected from a blood supply or of tissue, such as internal organs that are connected to a blood supply.

Human tissue, as an example, includes a variety of light-absorbing molecules referred to as chromophores. Such chromophores in the interior tissue of a body include oxygenated hemoglobin, deoxygenated hemoglobin, water, lipid, and cytochrome. Oxygenated hemoglobin and deoxygenated hemoglobin are the most dominant chromophores in interior tissue for much of the visible and near-infrared spectral range. Light absorption differs significantly for oxygenated and deoxygenated hemoglobins at certain wavelengths of light. Tissue oximeters can measure oxygen levels in human tissue by exploiting these light-absorption differences.

Despite the success of existing oximeters, there is a continuing desire to improve oximeters by, for example, improving form factor; allowing for modularity where one or more modules may be reusable and one or more other modules may be disposed of after use and replaced with a new one of the modules; improving measurement accuracy; reducing measurement time; lowering cost; reducing size, weight, or form factor, such as for; reducing power consumption; and for other reasons, and any combination of these measurements.

Therefore, there is a need for improved tissue laparoscopic oximeters and methods of making measurements using these probes.

### BRIEF SUMMARY OF THE INVENTION

A laparoscopic oximeter includes an oximeter sensor at its tip, which allows the making of oxygen saturation measurements laparoscopically. The laparoscopic oximeter includes a reusable probe unit and a laparoscopic tube that detachably connects to the probe unit so that the laparoscopic tube can be replaced for different patient surgeries and the probe unit can be reused for the different surgeries. Replaceable laparoscopic tubes facilitate efficient sterility for patient surgeries and a reusable probe unit facilitates cost savings and ecological conservation for a probe unit that includes costly circuitry.

In the separable design, the laparoscopic tube includes an optical coupler for coupling the optical fibers of the probe unit and the laparoscopic tube, where the optical fibers have different numerical apertures to increase the repeatability of coupling the optical fibers so that repeatable optical transfer efficiency is provided for more accurate oximetry measurements even when the connecting tips of the optical fibers are misaligned.

The probe unit and laparoscopic tube include a number of alignment systems to align the tips of the source optical fibers of the probe unit and the laparoscopic tube so that the tips connect when the probe unit and laparoscopic tube are connected.

A first alignment system of the number of types of alignment systems includes a key slot located in a collet and a first key located on an optical coupler where the first key in the key slot sets a rotational orientation of the collet with respect to the optical coupler and a first number of optical fibers that include ends of the optical fibers housed by the optical coupler.

A second alignment system of the number of types alignment systems includes the key slot and a second key located on the probe unit where the second key in the key slot sets a rotational orientation of the collet, the optical coupler, and the first optical fibers with respect to the probe unit, second optical fibers of the probe unit, and photodetectors of the probe unit.

A third alignment system of the number of alignment systems includes a number of ridges formed on an inside surface of a tubular portion of the collet and a round outer surface (such as a cylindrical surface) of the optical coupler. When the ridges contact the outer surface of the optical coupler, a first distance is set between a central axis of the collet to a central axis of the optical coupler.

A fourth alignment system of the number of alignment systems includes the number of ridges formed on an inside surface of a tubular portion of the collet and an outer surface of a tube portion (e.g., the cylindrical outer surface of the baffle cap) of the probe unit. When the ridges contact the outer surface of the baffle cap portion of the probe unit, a second distance is set between the central axis of the collet to a central axis of the tube portion of the probe unit. A distance between the centers of each of the connected tips of the first optical fibers and the tips of the second optical fibers is based on the first and second distances, such as a difference between the first and second distances. The difference between the first and second distances is less than a diameter of each second optical fiber.

The laparoscopic tube includes a communication tag, such as a near field communication (NFC) tag, that stores calibration information for the optical elements, such as optical fibers, housed by the tube. The probe unit includes a communication tag reader, such as an NFC tag reader, that can retrieve the stored calibration information from the NFC tag. The probe unit includes a memory that stores calibration information. Stored calibration information is used by the processor of the medical device to calibrate generated oximetry information when the medical device is used. In an embodiment, a memory in the laparoscopic tube is electrically connected to the probe unit so that information in the memory can be read and used by the probe unit. The information stored in the memory can be the same information that is stored in the communication tag, such as calibration information, an identifier, an expiration date, other information, or any combination of this information.

Other objects, features, and advantages of the present invention will become apparent upon consideration of the following detailed description and the accompanying drawings, in which like reference designations represent like features throughout the figures.

### BRIEF DESCRIPTION OF THE DRAWINGS

FIG. 1 shows a block diagram of a laparoscopic oximeter, in an implementation.

FIGS. 2A-2D are block diagrams of an oximeter probe, in various implementations.

FIGS. 3A-3B show diagrams of a laparoscopic oximeter, in an implementation.

FIG. 4A shows one of the source optical fibers of the probe unit that is connected tip-to-tip to one of the source optical fibers of the laparoscopic tube, in an implementation.

FIG. 4B shows a cross-sectional view of one of the cores of a source optical fiber of the probe unit that is connected tip-to-tip to one of the source optical fibers of the laparoscopic tube, in an implementation.

FIG. 4C shows one of the source optical fibers of the probe unit that is connected tip-to-tip to one of the source optical fibers of the laparoscopic tube, in an implementation.

FIG. 4D shows a cross-sectional view of one of the cores of a source optical fiber of the probe unit that is connected tip-to-tip to one of the source optical fibers of the laparoscopic tube, in an implementation.

FIGS. 5-6, respectively, show a perspective view and a planar view of a laparoscopic oximeter, in an implementation.

FIGS. 7A-7B show side views of the laparoscopic oximeter with a removable battery housing connected to the probe unit.

FIGS. 8-9, respectively, show a perspective view of the probe unit and a perspective view of a laparoscopic tube, in an implementation.

FIGS. 10A-10B show end views of the laparoscopic tube.

FIG. 11 shows an end view of the laparoscopic tube where the sensor head is located in the elongated tube of the laparoscopic tube.

FIGS. 12-13 show cross-sectional views of the laparoscopic tube, in an implementation.

FIG. 14 shows a cross-sectional view of the threaded locking cap of the laparoscopic tube.

FIG. 15 shows a cross-sectional view of one of the inner housings of the laparoscopic tube.

FIGS. 16-17 show the outside surface of the collet of the laparoscopic tube.

FIGS. 18-19 show a perspective end view of the collet of the laparoscopic tube.

FIG. 20 shows a transparent view of the optical coupler of the laparoscopic tube, in an implementation.

FIG. 21 shows a perspective view of the probe unit of the laparoscopic oximeter, in an implementation.

FIG. 22 shows the baffle cap of the probe unit attached to the collet of the laparoscopic tube.

FIG. 23 shows the optical coupler of the laparoscopic tube butted against the baffle cap of the probe unit.

FIG. 24 shows a cross-sectional view of the probe unit coupled to the laparoscopic tube, in an implementation.

FIG. 25 shows a perspective view of the probe unit with the threaded cap and baffle cap removed from the probe unit, in an implementation.

FIGS. 26-27, respectively, show the light engine of the probe unit connected to the source optical fibers of the probe unit and an exploded view of these elements.

FIGS. 28-29, respectively, show a perspective view and a side vice of the source optical fibers of the laparoscopic tube tip-to-tip connected to the source optical fibers of the probe unit.

FIGS. 30A-30B, respectively, show front and back views of a first PCB of the probe unit on which the photodetectors are mounted.

FIG. 31 shows the laparoscopic oximeter configured to transmit oximetry information to a remote display via a communication link for the display of the oximetry information on the display.

FIG. 32 shows the display connected to the laparoscopic tube by a detachable cable, in an implementation.

### DETAILED DESCRIPTION OF THE INVENTION

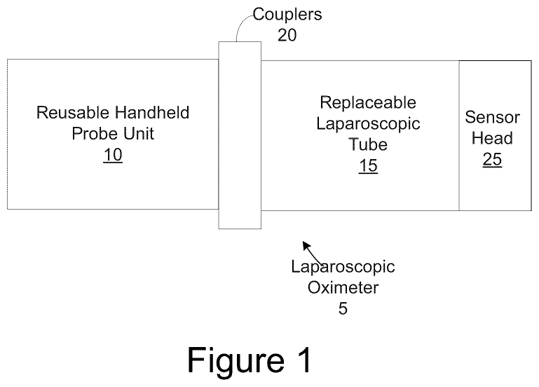

FIG. 1 shows a block diagram of a laparoscopic oximeter 5, in an implementation. Laparoscopic oximeter 5 includes a probe unit 10, an elongated tube 15, and couplers 20. The elongated tube can be a laparoscopic tube for laparoscopic surgeries. The laparoscopic tube includes a first end and a second end that are located at opposite ends of the laparoscopic tube. At least one of the couplers 20 is located at the first end of the laparoscopic tube and a sensor head 25 is located at the second end of the laparoscopic tube. The probe unit includes an end at which another one of the couplers 20 is located.

Laparoscopic oximeter 5 is configured to make oximetry measurements of tissue, such as internal tissue, intraoperatively. Laparoscopic oximeter 5 can be a tissue oximeter adapted to make tissue oximetry measurements or a pulse oximeter adapted to make pulse oximetry measurements.

Laparoscopic oximeter 5 is a handheld device where laparoscopic tube 15 extends from probe unit 10. In an implementation, the laparoscopic tube of the laparoscopic oximeter is adapted for intraoperative use in a patient and can be introduced into the abdominal cavity of the patient through a trocar. An outside surface of the laparoscopic tube can be smooth so that the laparoscopic tube can slide through the trocar smoothly, can rotate within the trocar smoothly, and can slide into contact and past patient tissue smoothly and without abrading the tissue. The laparoscopic oximeter can be used on various internal tissues to determine various oximetry information for the tissue, such as oxygen saturation. Internal tissue under test can include intestinal tissue, such as the large intestine, small intestine, tissue that supports these tissues, such as the mesentery tissue, muscle, the liver, kidneys, stomach, gallbladder, pancreas, arteries, heart, lungs, veins, or other internal tissue.

The laparoscopic oximeter is fully self-contained and does not need to be connected to another device to be fully operational, in an implementation. That is, the laparoscopic oximeter does not need to be wire connected or wirelessly connected to another device to operate. In an implementation, the laparoscopic oximeter does connect to other devices, such as one or more other medical devices, a computer system, a display, these devices or systems, or other devices or systems.

Couplers 20 include mechanical couplers that allow for the probe unit and laparoscopic tube to be mechanically connected and disconnected. The couplers include optical couplers that allow for the probe unit and the laparoscopic tube to be optically connected and optically disconnected, in an implementation. The optical couplers allow for light to be transmitted from the probe unit to the laparoscopic tube, from the laparoscopic tube to the probe unit, one of these directions of light travel, or both of these directions of light travel. The couplers can include electrical couplers that allow the probe unit and laparoscopic tube to be electrically connected and electrically disconnected in an implementation. The electrical couplers allow for electrical signals to be transmitted from the probe unit to the laparoscopic tube, from the laparoscopic tube to the probe unit, one of these directions, or both of these directions. The couplers can include inductive couplers that allow the probe unit and laparoscopic tube to be inductively coupled by radio frequency alternating magnetic fields.

In an implementation, the couplers facilitate the separation of the probe unit from the laparoscopic tube. For example, the probe unit and laparoscopic tube can be separated after the laparoscopic oximeter has been used for a laparoscopic surgery. The probe unit can be reused after the surgery and the laparoscopic tube can be discarded. The probe unit can be cleaned after the surgery and connected to a different laparoscopic tube to form another laparoscopic oximeter. The probe unit can thereafter be used in a different surgery. The probe unit is sometimes referred to as a durable unit because this unit is reusable, whereas the laparoscopic tube is sometimes referred to as a disposable unit because it is to be disposed of after use on a patient.

In an implementation, the probe unit includes various components that are relatively costly. Allowing for the probe unit to be reused allows for cost savings by the reuse. Allowing for the probe unit to be reused also allows for ecological advantages because the probe unit can be reused a number of times before the probe unit is disposed of.

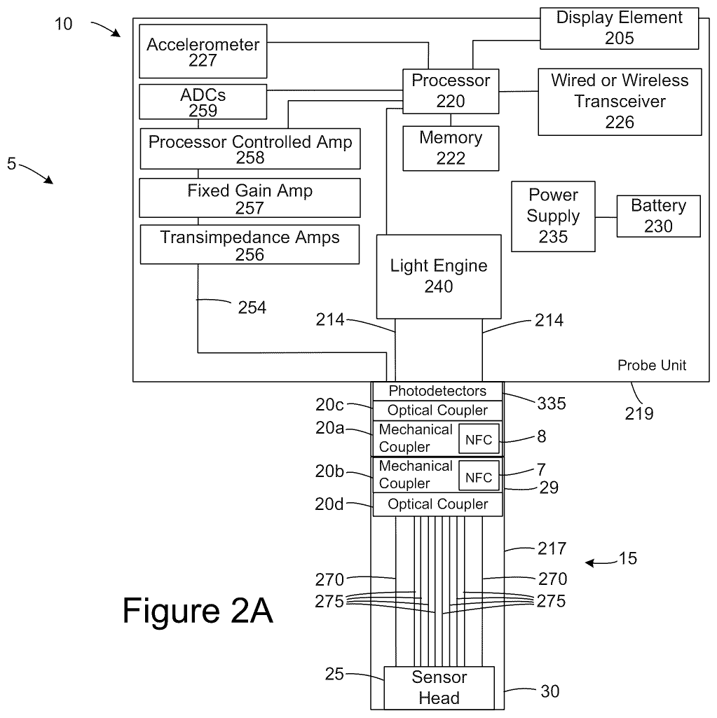

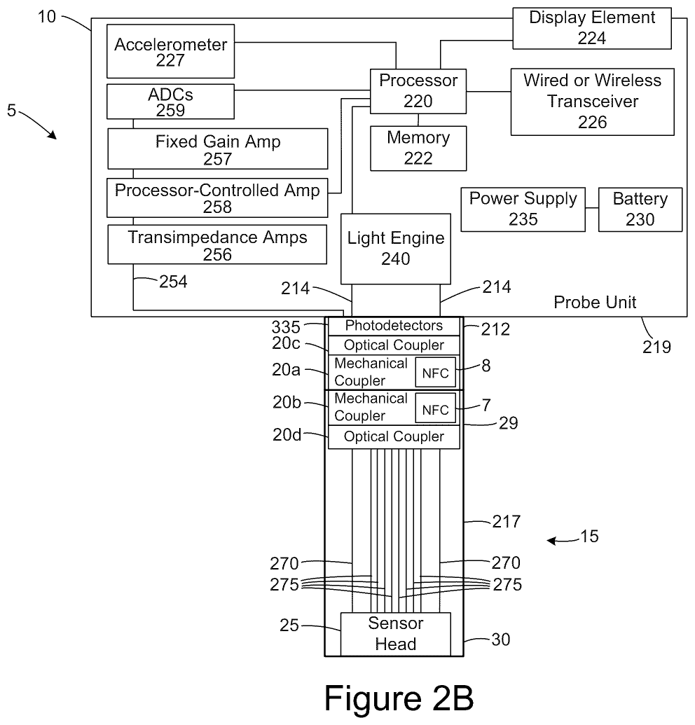

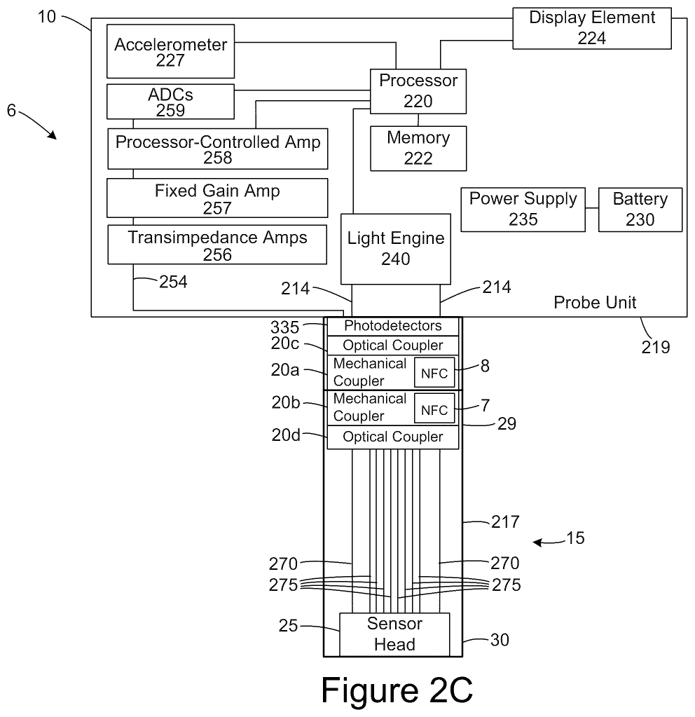

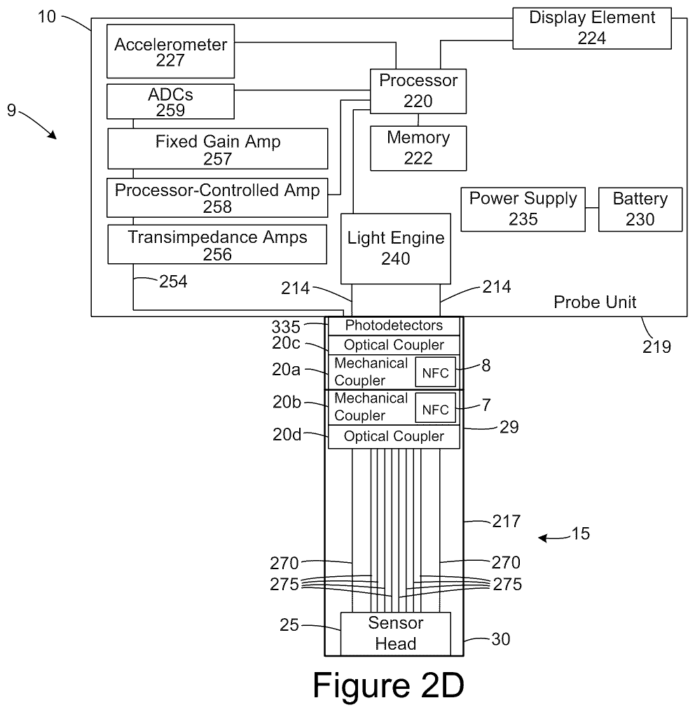

FIG. 2A shows a diagram of laparoscopic oximeter 5, in an implementation. The probe unit 10 of laparoscopic oximeter 5 includes a housing 219 that houses a processor 220, a memory 222, a display 205, a transceiver 226, an accelerometer 227, a battery 230, a power supply circuit 235, a light engine 240, one or more transimpedance amplifiers (TIAs) 256, one or more fixed gain amplifiers 257, one or more processor-controlled amplifiers 258, one or more analog-to-digital converters (ADCs) 259, at least portions of one or more electrical conductors 254 (e.g., wires in a cable or electrical traces in a printed circuit board), at least portions of one or more source waveguides 214 (e.g., source optical fibers), other elements, or one or more of these elements in any combination.

The battery is connected to the power supply circuit, which is connected and supplies battery power to the processor, memory, display, transceiver, accelerometer, light engine, the one or more amplifiers, and the one or more analog-to-digital converters. The battery of the probe unit can include one or more of a variety of battery types, such as one or more disposable batteries or one or more rechargeable batteries. Disposable batteries are discarded after their stored charge is expended. Some disposable battery chemistry technologies include non-rechargeable lithium ion, alkaline, zinc carbon, or silver oxide. The battery has a sufficient stored charge to allow the use of the handheld device for several hours.

In an implementation, the battery, the power supply circuit, or both are housed in a detachable housing that can be detachably connected to housing 219. The detachable housing is described further below. The battery housing can be replaced by another battery housing when the battery discharges.

In implementations where the battery is rechargeable, the battery can be recharged multiple times after the stored charge is expended. Some rechargeable battery chemistry technologies include nickel cadmium (NiCd), nickel metal hydride (NiMH), lithium ion (Li-ion), and zinc air. The battery can be recharged, for example, via a wireless charging system (e.g., a wireless charging system operating according to the Qi standard or another charging standard), via an AC adapter with a cord that connects to the handheld unit. The circuitry in the display can include a recharger circuit (not shown). In another implementation, the batteries are disposable batteries that cab be disposed of and replaced once the charge of the batteries is expended.

The processor is connected to the memory, display, transceiver, accelerometer, light engine, the ADCs, and the one or more amplifiers. The ADCs are connected between the amplifiers and the processor. The amplifiers may be the TIAs, the fixed gain amplifier, the processor-controlled amplifier, or any of these amplifiers in any combination.

In the implementation shown in FIG. 2A, for example, the transimpedance amplifier 256 is connected to photodetectors 335 (described further below) via one or more of the electrical conductors 254. The fixed gain amplifier is connected to both the transimpedance amplifier and the processor-controlled amplifier and is connected between the transimpedance amplifier and the processor-controlled amplifier. The processor-controlled amplifier is connected to the processor for processor-controlled amplification of the voltage amplification by the processor. The processor-controlled amplifier is also connected to the ADCs.

In an implementation shown in FIG. 2B, for example, the transimpedance amplifier 256 is connected to the photodetector 335 via the one or more electrical conductors 254. The processor-controlled amplifier is connected to both the transimpedance amplifier and the fixed gain amplifier and is connected between the transimpedance amplifier and the fixed gain amplifier. The processor-controlled amplifier is also connected to the processor for processor control of the voltage amplification of this amplifier. The fixed gain amplifier is connected to the ADCs.

The TIAs may amplify a low current signal generated by the photodetectors to a stable voltage signal. The fixed gain amplifier may amplify the voltage output from the TIAs to a higher voltage. The processor-controlled amplifier may amplify the higher voltage output by the fixed gain amplifier to a voltage usable by the ADC.

The light engine is electrically connected to the processor and the processor controls the generation of light by the light engine for emission of the light, such as emission into patient tissue or a tissue proxy when the probe unit is used. The light engine includes one or more sources that generate and transmit light, such as visible light, infrared light, or both. The light engine may include one ore more LED driver circuits that are coupled between the processor and the source. The LED driver circuit can drive the sources at a duty cycle that can be varied to adjust the intensity of emitted light. The LED drive circuit can drive the sources at a substantially fixed current when the sources are powered on. The substantially fixed current can vary by an amount that will be understood by those of ordinary skill in the art to maintain a substantially constant light emittance by the sources. For example, the current may vary by up to 1 percent to maintain a relatively constant light emittance.

Each source can include one or more light emitting diodes (LEDs), such as one, two, three, four, five, six, seven, eight, nine, ten, or more LEDs. Each LED is adapted to emit one or more wavelengths of light, such as visible light, infrared light, or both. In one implementation, the LEDs emit wavelengths of 760 nanometers, 810 nanometers, and 845 nanometers. In another embodiment, the LEDs emit wavelengths of 760 nanometers, 810 nanometers, 845 nanometers, and 895 nanometers. The wavelengths can vary by plus or minus a nanometer.

The LEDs can be discrete LEDs, organic LEDs (OLEDs), high brightness LEDs (HLEDs), quantum dot LEDs, laser diodes, or other types of LEDs.

The LEDs are optically connected to the first ends of one or more source waveguides 214. The LEDs can be optically connected to the first ends of the one or more source waveguides inside of housing 219. Second ends of the source waveguides are connected to a first optical coupler 20c of the probe unit. In an implementation, the first optical coupler includes a number of apertures formed in the coupler and portions of the second ends of the source waveguides are located in the apertures. Tip portions of the second ends of the source waveguides can extend from the apertures so that the second ends extend outside of the apertures. The tip portions are the terminal ends of the source waveguides.

The source waveguides can be optical fibers or other light-guiding elements. One, two, three, four, five, or more LEDs can be optically connected to one or more source waveguides 214. The one or more source waveguides can include 1, 2, 3, 4, 5, 6, 7, 8, 9, 10, or more waveguides.

In an implementation, couplers 20 and probe unit 10 include a mechanical coupler 20a and the optical coupler 20c. Probe unit 10 can further include a tag reader 8, one or more photodetectors 335, portions of one or more wires 254, one or more source optical fibers 214 (e.g., two), other elements, or any combination of these elements. The couplers 20a and 20c, the tag reader 8, and the photodetectors 335 are located outside of housing 219, in one implementation of the laparoscopic oximeter. In an alternative implementation, housing 219 houses the couplers 20a and 20c, the tag reader 8, and the photodetectors 335.

In an implementation, couplers 20 and laparoscopic tube 15 include a mechanical coupler 20b and an optical coupler 20d. Laparoscopic tube 15 can further include an elongated tube 217, a communication tag 7, one or more source waveguides 270 (e.g., two), one or more detector waveguides (e.g., eight), and the sensor head 25. Source waveguides 270 and detector waveguides 275 can be source optical fibers and detector optical fibers, respectively.

The mechanical coupler 20b and the optical coupler 20d are located at a first end 29 of the laparoscopic tube and the sensor head 25 is located at a second end 30 (e.g., a tip) of the laparoscopic tube. The first and second ends of the laparoscopic tube are located at opposite ends of the tube. The mechanical and optical couplers are located outside of elongated tube 217 at a first end of the laparoscopic tube, in an implementation. In an alternative implementation, one or both of the mechanical and optical couplers are located inside of elongated tube 217.

In an implementation, the sensor head 25 is located inside the tip 30 of elongated tube 217 where the housing has an opening from which the sensor head can emit light and receive light. In an implementation, the sensor head is completely housed within the interior space of the tip of the housing.

In an implementation, the laparoscopic tube is a passive optical device and does not include any electrical or electronic elements. The source waveguides 270 and the detector waveguides 275 extend from optical coupler 20d to sensor head 25. The first ends of the source waveguides can located in apertures formed in the optical coupler and second ends of the source waveguides can be located in apertures formed in the sensor head so that light traveling through the waveguides can be emitted from the sensor head and from the laparoscopic oximeter.

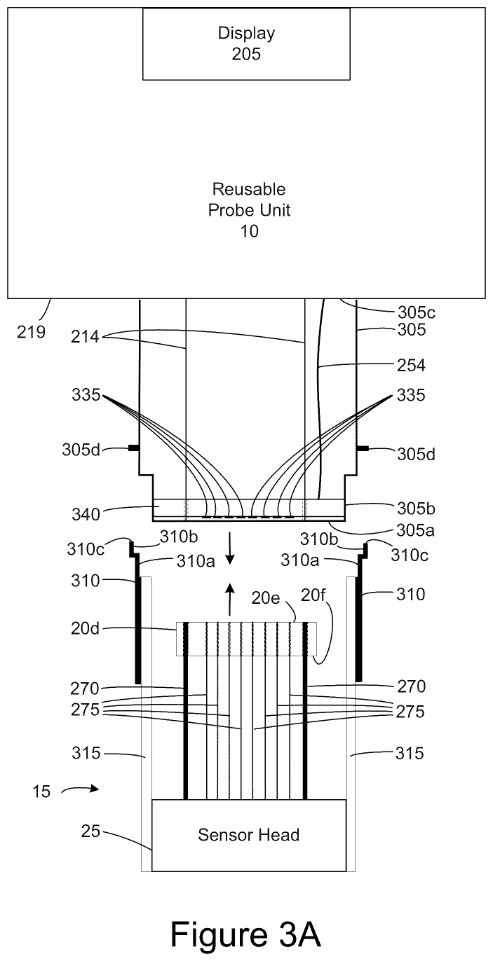

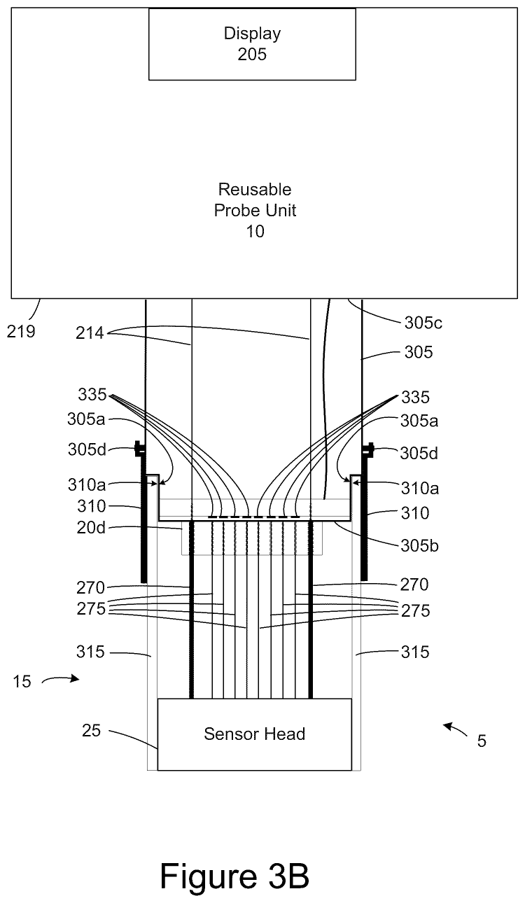

Detector waveguides 275 extend from optical coupler 20d to sensor head 25. The first ends of the detector waveguides can be located in apertures formed in the optical coupler and the second ends of the detector waveguides can be located in apertures formed in the sensor head so that light emitted from the source waveguides and reflected from patient tissue or a calibration block of material, for example, can be received by the second ends of the detector waveguides and transmitted to the first ends of the fibers. The apertures for the source and detector waveguides are shown with dashed lines in FIGS. 3A-3B, which are described below.

For example, the light can be emitted into patient tissue when an oximetry measurement of the tissue is being performed by the laparoscopic oximeter or can be emitted into a calibration block of material when the laparoscopic oximeter is being calibrated. The light can be received by the detector waveguides after the light is reflected from patient tissue, a calibration block, or other material. The received light can be transmitted by the detector waveguides to the photodetectors of the probe unit. Additional operation of the laparoscopic oximeter is described below.

In an implementation, the diameters of the apertures in optical coupler 20d for the source waveguides 270 are larger than the diameters for the apertures for the detector waveguides 275. The apertures for the source waveguides can have approximately equal diameters. The apertures for the detector waveguides can also have approximately equal diameters. In some implementations, the apertures for the detector waveguides have different sizes to accommodate detector waveguides that have different diameters. For example, the detector waveguides might have different diameters when a larger diameter waveguide is used at a distance to collect more light than collected by the smaller diameter detector waveguide.

Source waveguides 270 of the laparoscopic tube are optically coupled, respectively, to source waveguides 214 of the probe unit. Optical couplers 20c-20d optically couple source waveguides 270 and source waveguides 214 when mechanical couplers 20a-20b are connected. When the optical couplers connect source waveguides 214 to source waveguides 214, light generated by light engine 240 can travel from source waveguides 214 to source waveguides 270. The light can be emitted from source waveguides 270 at the sensor head.

The first ends of detector waveguides 275 of the laparoscopic tube are optically coupled to the photodetectors when mechanical couplers 20a-20b are connected. Light received by the detector waveguides at the sensor head at the second ends of the waveguides is transmitted through the waveguides to the first ends to the photodetectors.

When the photodetectors convert received light into electrical signals, the TIAs 256 amplify the electrical signal. Each photodetector may be electrically connected to one TIA by wires 254. In an implementation, multiple photodetectors are connected to a single TIA, for example, via a multiplexer. The TIAs are adapted to receive analog detector responses generated by the photodetectors. The TIAs convert the current for the analog detector responses to a voltage and amplify the voltage. The fixed gain amplifier 257 and the processor-controlled amplifier, respectively, amplify the voltage signals to levels that the ADCs can convert to digital signals that are used by the processor.

The ADCs sample the analog signals at a sampling rate. For example, the sampling rate can be about on the order of kilohertz, such as about 200-300 kilohertz. The measurement rate that the processor operates on the digitized signals is less than the sampling rate. The measurement rate that the processor operates on the digitized signals is about 1-3 hertz (depending on the conditions of the oximeter probe). In other implementations, the measurement rate can be above 3 hertz, such as from about 4 hertz to about 1 kilohertz. Generally, the faster the sampling rate of the ADCs and the operating frequency of the processor, the more power that is consumed, which is a consideration for a battery-operated device, and also the data generated increases with the sample rate.

When using a measurement rate of about 0.33 to about 3 hertz, the amount of data can be transmitted wirelessly by the transceiver to other devices (e.g., a computer or a display) using technologies such as Bluetooth and Wi-Fi (and others mentioned in this patent) without data loss. In other implementations, a proprietary wireless technology can be used, such as when higher sampling rates are desired.

The processor can apply one or more calibration calculations to digitized detector responses received from the ADCs, such as performing one or more calibration steps on the data. Calibration can include calibrating the digitized detector responses for inherent discrepancies in the intensity of light emitted by the LED of the light engine, for inherent discrepancies in the detection sensitivity of the photodetectors, or both, or other correlation calculations. Calibration information for the LEDs and photodetectors can be predetermined and stored in the laparoscopic oximeter's memory.

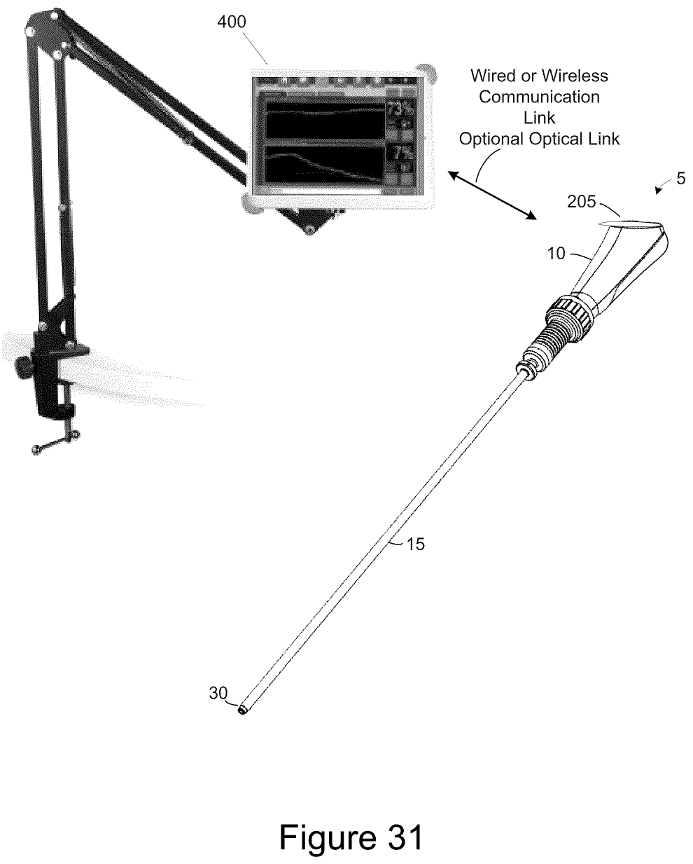

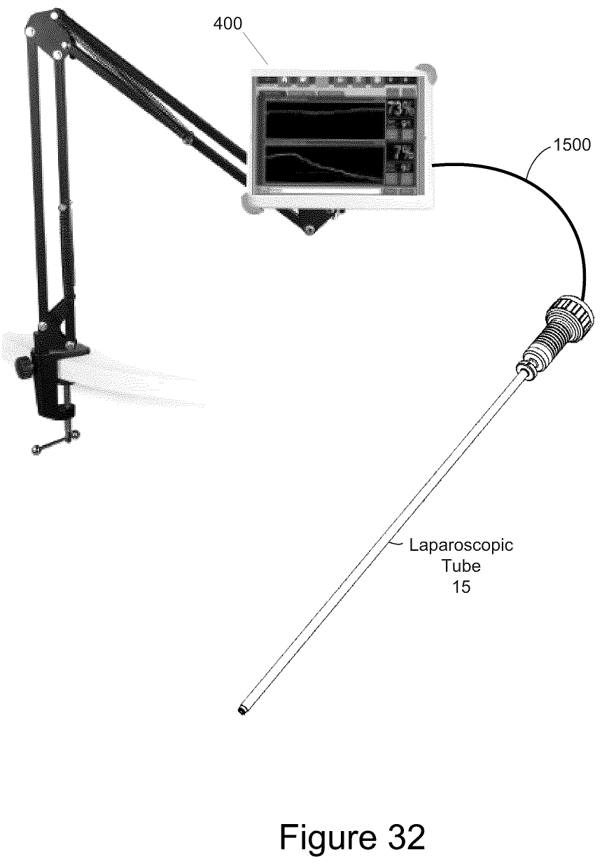

As part of the data acquisition process for oximetry measurements performed by the processor of the oximeter device or display 400 (FIGS. 31-32), the image noise in the oximetry measurement values is reduced by one or more dark subtraction techniques. Image noise is noise in the signal data that is generated by the oximeter where the noise is due to light that the photodetectors of the oximeter detect but that is not transmitted by the oximeter into patient tissue, is due to noise that the photodetectors of the oximeter generate without detecting light, is due to electrical noise in the oximeter, such as noise from electrical crosstalk, is due to optical noise in the oximeter, such as noise from optical crosstalk, or includes noise from other noise sources in the oximeter. Image noise lowers the signal-to-noise ratio of oximetry measurement values, such as oxygen saturation values, absorption coefficient values, reflection coefficient values, or other measured or calculated values.

In an implementation, after the photodetectors detect reflected light or transmitted light from patient tissue for an oximetry measurement and after the oximeter device stops emitting light, a noise data sampling is taken by the photodetectors. More specifically, during the noise data sampling, the light sources of the oximeter device do not generate light or light generated by the light sources is not emitted from the device.

The processor then collects the values for the noise data samples. Thereafter, the values for the noise data samples can be subtracted from the values for the data samples taken for one or more oximetry measurements, such as an oximetry measurement immediately preceding the collection of the noise data samples. Alternatively, the values for the noise data samples can be subtracted from the values for data samples taken for a number of preceding oximetry measurements.

In an implementation, the values for the noise data generated by a given photodetector are respectively subtracted by the processor from the values for oximetry measurements generated by the photodetector. The subtraction step can be performed by the processor for each photodetector. The resultant oximetry measurement values (also sometimes referred to as corrected oximetry measurement values) may then be used by the processor to generate further oximetry values, such as oxygen saturation values, absorption coefficient values, scattering coefficient values, other intermediary calculated values, or other final calculation values.

In another implementation, the values for the noise data samples generated by the photodetector are averaged and subtracted by the processor from the values for the oximetry measurements. The resultant oximetry measurement values may then be used by the processor to generate further oximetry values, such as oxygen saturation values, absorption coefficient values, scattering coefficient values, other intermediary calculated values, or other final calculated values.

Dark subtraction can be performed for one or more combinations of sources and detectors. For example, where the oximeter includes 4 light sources (e.g., LEDs) and 2 photodetectors, dark subtraction can be performed for 8 data samples for 8 oximetry measurements for each combination of light sources and photodetectors. The eight combinations of 4 light sources (S1, S2, S3, S4) and 2 photodetectors (D1 and D2) includes S1-D1, S2-D1, S3-D1, S4-D1, S1-D2, S2-D2, S3-D2, and S4-D2.

For example, for a value for an oximetry measurement generated for a D1 response to light emitted by S1, a noise data sample may be taken for D1, and the value for the noise data sample may be subtracted from the value for the oximetry measurement. For a value for an oximetry measurement generated for a D1 response to light emitted by S2, a noise data sample may be taken for D1, and the value for the noise data sample may be subtracted from the value for the oximetry measurement. For a value for an oximetry measurement generated for a D1 response to light emitted by S3, a noise data sample may be taken for D1, and the value for the noise data sample may be subtracted from the value for the oximetry measurement. For a value for an oximetry measurement generated for a D1 response to light emitted by S4, a noise data sample may be taken for D1, and the value for the noise data sample may be subtracted from the value for the oximetry measurement.

For a value for an oximetry measurement generated for a D2 response to light emitted by S1, a noise data sample may be taken for D2, and the value for the noise data sample may be subtracted from the value for the oximetry measurement. For a value for an oximetry measurement generated for a D2 response to light emitted by S2, a noise data sample may be taken for D2, and the value for the noise data sample may be subtracted from the value for the oximetry measurement. For a value for an oximetry measurement generated for a D2 response to light emitted by S3, a noise data sample may be taken for D2, and the value for the noise data sample may be subtracted from the value for the oximetry measurement. And, for a value for an oximetry measurement generated for a D2 response to light emitted by S4, a noise data sample may be taken for D2, and the value for the noise data sample may be subtracted from the value for the oximetry measurement.

The resultant values for the oximetry measurements that are corrected for image noise may thereafter be used by the processor to generate oximetry measurement information, such as an oxygen saturation value, an absorption coefficient value, a reflection coefficient value, or other values generated by the oximeter device.

The values for the noise data sample can include raw data or processed values. Raw data can include data that is digitized, amplified, or both, but otherwise not processed by the processor.

In an implementation, values for image noise samples can collected during the manufacture of the oximetry device. During manufacture, the values for the image noise samples can be for image noise generated when the oximeter device is not emitting light and can be a measure of electrical crosstalk or other electrical noise in the oximeter device. During manufacture, the values for the image noise samples can be for image noise generated when the oximeter device is generating light but when a target is not placed in front of the probe tip of the oximeter device. The values for the image noise samples can be a measure noise of both electrical crosstalk and optical crosstalk in the oximeter device. One or both of the values for noise data samples (generated with or without light transmission) can be stored in a memory of the oximetry device. The stored values for the noise data samples can be used by the oximeter device for dark subtraction when the oximeter device is last used to make patient oximetry measurements.

In an implementation, the processor of the oximeter device operates according to a cross-correlation of inphase\quadrature signal components (such as signal components for the image noise signal) method to reduce image noise for values for oximetry measurements.

The processor is adapted to use spatially resolved spectroscopy techniques for determining oximeter information, such as blood oxygen saturation, of tissue from the detector signals that are digitized by the ADCs. Spatially resolved spectroscopy is facilitated by the distances between the source structure and the detector structure of the sensor head.

The following U.S. patent applications are incorporated by reference along with all other references cited in this application: Ser. Nos. 13/887,130, 13/887,220, 13/887,213, 13/887,178, and 13/887,152, filed May 3, 2013; Ser. No. 13/965,156, filed Aug. 26, 2013; Ser. No. 15/493,132, 15/493,111, 15/493,121, filed Apr. 20, 2017; Ser. No. 15/494,444, filed Apr. 21, 2017; and Ser. Nos. 15/495,194, 15/495,205, and 15/495,212, filed Apr. 24, 2017. U.S. patent application Ser. Nos. 19/012,790, 19/012,792, 19/012,826, and 19/012,833, filed Jan. 7, 2025, are also incorporated by reference. The above applications describe various laparoscopic oximeters and oximetry operations, such as spatially resolved spectroscopy, and discussion in the above applications can be combined with aspects of the invention described in this application, in any combination.

In an implementation, the elongated tube 217 of the laparoscopic tube can be plastic, a plastic-type material, or metal, such as stainless steel or titanium. The housing 219 of the probe unit can be plastic, a plastic-type material, or metal, such as stainless steel or titanium. The laparoscopic tube has a length of 10 centimeters to 60 centimeters. More specifically, the tip of the sensor head can be one of a variety of distances (e.g., 10 centimeters to 60 centimeters) from the top of the laparoscopic tube where the length can be based on the particular medical procedure that the laparoscopic oximeter is used for. The outside cross-sectional dimension of the elongated tube 217 of the laparoscopic tube can range from about 3 millimeters to about 20 millimeters or greater (e.g., 3, 4, 5, 6, 7, 8, 9, 10, 15, or 10 millimeters, or others). The cross-sectional shape of the elongated tube of the laparoscopic tube can be, for example, circular, elliptical, oval, or any rounded polygon (e.g., square or rectangle with rounded corners).

Optical fibers 270 located in laparoscopic tube 15 can have a variety of lengths, such as from about 10 centimeters to about 60 centimeters. Optical fibers 214 located in probe unit 10 can have a variety of lengths, such as from about 2 centimeters to about 10 centimeters. Each optical fiber 270 is longer than each optical fiber 214. Each optical fibers 270 can be about 5 times to about 30 times longer than each optical fiber 214. Optical fibers 275 located in laparoscopic tube 15 can have a variety of lengths, such as from about 10 centimeters to about 60 centimeters. Optical fibers 276 located in probe unit 10 can have a variety of lengths, such as from about 2 centimeters to about 10 centimeters. Each optical fiber 275 is longer than each optical fiber 276. Each optical fiber 275 can be about 5 times to about 30 times longer than each optical fiber 214.

The outer face of the sensor head of the laparoscopic tube can have a variety of shapes, such as flat or rounded. In an implementation, the outer face of the sensor head of the laparoscopic tube is not transverse with respect to the lateral cross-section of the elongated tube of the laparoscopic tube. In other embodiments, the outer face of the sensor head is flush with the end of the elongated tube of the laparoscopic tube, recessed within the elongated tube, or extends from the elongated tube. The outside surface, inside surface, or both outer and inside surfaces of elongated tube 217 of the laparoscopic tube can also be coated, such as with a polymeric coating, to make the one or more surfaces nonconducting.

Mechanical couplers 20a-20b can include one or more coupler types, including spring couplers, threaded couplers, mechanical latches, magnetic couplers, pins (retractable pins that are spring-loaded to push the pins out from a pushed-in configuration) and apertures to receive the pins, or other couplers. One or both of mechanical couplers 20a-20b can be keyed so that the mechanical couplers mate and couple in one orientation with respect to each other. Specific implementations of mechanical couplers 20a-20b are described further below with respect to specific embodiment of the laparoscopic oximeter.

Communication tag 7 can be a radio frequency identification tag, such as a near field communication (NFC) tag, The tag reader 8 can be a radio frequency identification tag reader, such as an NFC tag reader. Communication tag 7 can be coupled to mechanical coupler 20b so that the communication tag is near the first end 29 of the laparoscopic tube. The tag reader 8 can be coupled to the mechanical coupler 20a so that the tag reader is near the first end 29 of the laparoscopic tube and near communication tag 7 when the probe unit and the laparoscopic tube are coupled by mechanical couplers 20a-20b. When the probe unit and the laparoscopic tube are connected, tags 7 and 8 are positioned relatively near to each other so that the communication tag and tag reader can communicate via radio frequency changing magnetic fields.

FIG. 2C shows an oximeter probe 6, in an implementation. Oximeter probe 6 differs from oximeter probe 5 shown in FIG. 2A and described above in that oximeter probe 6 does not include a wired or wireless transceiver. FIG. 2D shows an oximeter probe 9, in an implementation. Oximeter probe 9 differs from oximeter probe 5 shown in FIG. 2B and described above in that oximeter probe 9 does not include a wired or wireless transceiver.

FIGS. 3A-3B show diagrams of laparoscopic oximeter 5, in an implementation.

FIG. 3A shows the laparoscopic oximeter 5 with the probe unit 10 detached from the laparoscopic tube 15 and FIG. 3B shows the probe unit connected to the laparoscopic tube.

Laparoscopic tube 15 includes a housing 310 and an elongated tube 315. Both housing 310 and elongated tube 315 are generally tubular shaped and have interior spaces in which elements of the laparoscopic tube are located. Housing 310 and elongated tube 315 are connected at the surface of an inner wall of the housing and at the surface of an exterior wall of the elongated tube. The elongated tube is about 2-20 times longer than the housing. Housing 310 and sensor head 25 are positioned at opposite ends (i.e., proximal and distal ends) of the elongated tube. The elongated tube is configured to be inserted in a trocar for insertion into a body cavity during a surgery. The elongated tube is sometimes referred to as a laparoscopic tube.

In an implementation, the probe unit includes a baffle cap 305. The baffle cap houses at least a portion of source waveguides 214 and houses at least a first printed circuit board (PCB) 340, which is located near an end of the baffle cap. In an implementation, the baffle cap includes one or more PCBs that are electrically coupled to PCB 340. The one or more PCBs located in the baffle cap include electrical traces that can transmit signals generated by the photodetectors to a set of wires 254

Photodetectors 335 are located on a side of PCB 340 that faces away from housing 219 and faces towards the laparoscopic tube when the laparoscopic tube is connected to the probe unit. The photodetectors are connected to the PCB 340 via solder connections, via solder connections and an adhesive (e.g., epoxy resin), or via solder connections and mechanically (e.g., a fastener, bracket, or another device).

The baffle cap can include a number of apertures formed in a bottom surface of the cap. The photodetectors are registered with the apertures so that light can pass through the aperture of the baffle cap to reach the photodetectors. In an implementation, at least a portion of the photodetectors is located in the apertures formed in the baffle cap. The one or more PCBs housed in the baffle cap include electrical traces that can transmit electrical signals generated by photodetectors 335 to the one or more electrical conductors 254. At least a portion of the electrical conductors can be located in the baffle cap.

The photodetectors can be one or more of a variety of detector types, such as photodiodes (e.g., a PN photodiode, a PIN photodiode, an avalanche photodiode, a Schottky photodiode, or other types of photodiodes), photoresistors, phototransistors, metal semiconductor-metal semiconductor devices, photoconductive detectors, phototubes, such as photoemissive detectors, phototubes, such as photomultipliers, carbon nanotubes (CNTs), or other types of photodetectors.

PCB 340 includes one or more apertures (e.g., two apertures) through which source waveguides 214 extend. The baffle cap also includes one or more apertures (e.g., two apertures) through which source waveguides 214 extend. The ends of source waveguides 214 can be flush with a bottom surface 305a of the baffle cap. Bottom surface 305a can be a flat surface that can be polished to flatten the surface. In an implementation, surfaces of the photodetectors are flush with bottom surface 305a.

In an implementation, optical coupler 20d includes a number of apertures formed in the coupler where the apertures extend from the top side 20e of the coupler to the bottom side 20f of the coupler. The number of apertures formed in the optical coupler equals the number of source and detector waveguides located in the laparoscopic tube. The ends of the source and detector waveguides are located in the apertures and can extend from the bottom side to the top sides of the optical coupler. The ends of the source waveguides, the detector waveguides, or both are flush with the top surface 20e of the optical. Top face 20e is a flat surface that can be polished to flatten the surface.

When the probe unit and laparoscopic tube are connected, an outside surface 305b of baffle cap 305 contacts an inside surface 310a of housing 310. The shapes of the outside surface and the inside surface are complementary shapes, such as circular, oval, square, rectangular, or other shapes. The contacting surfaces guide the bottom surface 305a of the baffle cap into contact with the top surface 20e of the optical coupler when the probe unit and laparoscopic tube are moved into contact. In an implementation where the outside and inside surfaces are circular, the outer diameter of the outside surface 305b a can be about 0.02-0.10 millimeters less than the inner diameter of the inside surface 310a.

The outside surface 305b and inside surface 310a can be keyed so that the probe unit can fit together with the laparoscopic tube in one orientation. The inner and outside surfaces can be keyed with a slot and a protruding key that fits into the slot when the probe unit and laparoscopic tube are slid together. The keyed connection facilitates the alignment of source waveguides 270 and 214, the alignment of the detector waveguides and the photodetector, or both. In an alternative implementation, the tip of the laparoscopic tube can slide into an opening of the baffle cap to couple the source waveguides and align the detector waveguides and the photodetectors.

The baffle cap includes a number of pins 305d that extend outward from an outer sidewall surface of the baffle cap. The baffle cap includes a corresponding number of apertures formed in the outer sidewall, where a portion of each pin is located in. The pins can be adhered in the baffle cap by friction fit, an adhesive, such as epoxy or other adhesive, or other connection devices. The number of pins includes two or more pins, such as three pins. In an implementation where the number of pins includes three pins, the pins can be arranged in an equilateral triangular configuration or can be arranged in an arrangement that is not an equilateral triangle. The pins are located in a plane that is parallel to the bottom surface 305a of the cap. In an implementation, the plane can be nearer to bottom surface 305a than to top 305c of the baffle cap. In another implementation, the plane is in other locations away from the bottom surface 305a. The plane can be from about 2 percent to about 20 percent of the length of the baffle cap from bottom surface 305a.

Housing 310 can include a number of slots 310b, such as slotted apertures, that are positioned to receive pins 305d into the slots. The number of slots equals the number of pins, such as three slots and three pins. The geometric arrangement of the slots and pins is the same. For example, in an implementation where the number of slots includes three slots, the slots and pins are arranged in equilateral triangular configurations. The slots are arranged in a plane that is parallel to an end 310c of housing 310.

Baffle cap 305 can be slid into contact with housing 310 and the pins can be correspondingly slid into the slots. The pins can be slid into the slots to facilitate the alignment of the tips of source waveguides 270 and 214 so that the tips align and contact one another. The pins and slots also facilitate the alignment of the tips of the detector waveguides in optical coupler 20d to the photodetector.

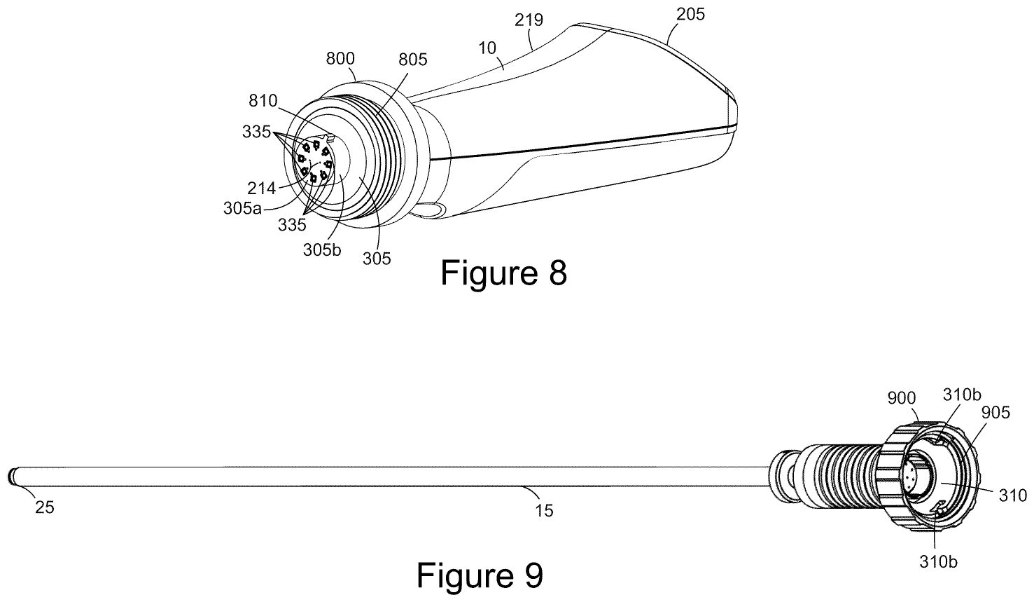

Slots 310b can have a shape, such as a “J” shape or other useful shapes such as a “7” shape. The shape allows the pins to be inserted into the slots, then rotated as the baffle cap is rotated with respect to housing 310, and then the pins enter a curved portion of the shape to removably lock the pins in the slots and removably lock the baffle cap to housing 310. The J shapes portion of the slots is described further below for a specific implementation of the slots. In an implementation, threaded sleeve 800 has an opening with an inner diameter and which an end portion of baffle cap 305 is located as shown in FIG. 8. Further, housing 310 has an outer diameter that is less than the inner diameter of threaded sleeve 800. The inner diameter of threaded sleeve 800 and the outer diameters of housing 300 allow for an end portion of housing 300 to enter the opening of threaded sleeve 800 so that pins 305d can enter the J shaped portions of the slots. Additionally, the baffle cap 305 has an outer diameter and housing 300 has an opening with an inner diameter so that the end portion of the baffle of the baffle cap can enter the opening of housing 300 so that pins 305d can slip into the J shaped portions of slots 310b when laparoscopic tube 15 is connected to the probe unit 10. The pins and slots can form a BNC type connection interface or a C type connection interface. In an implementation the coupling connection of baffle cap 305 and housing 310 can include spring-loaded F type connectors.

This detailed description describes examples of implementations with specific measurements, angles, values, dimensions, shapes, and orientations. These examples implementations are not intended to be exhaustive or to limit the invention to the precise form described.

The measurements, for example, in millimeters or centimeters are approximate values. The values can vary due to, for example, measurement or manufacturing tolerances (as will be understood by those of ordinary skill in the art) or other factors (as will be further understood by those of ordinary skill in the art). A measurement can vary, for example, by plus or minus 1 percent, plus or minus 5 percent, plus or minus 10 percent, plus or minus 15 percent, plus or minus 20 percent, plus or minus 1 to 5 percent, plus or minus 5 to 10 percent, or plus or minus 15 to 20 percent. Further, the measurements are for a specific implementation of the device, and other implementations can have different values, such as certain measurements, dimensions, or both made longer to accommodate smaller hands or larger hands or to access tissue in a particular location of a patient's body.

For the specific implementations described, some specific values, ranges of values, and numbers are provided. These values indicate, for example, dimension, angles, ranges, frequencies, wavelengths, numbers, a relationship (e.g., relative value), and other quantities (e.g., numbers of sensors, sources, detectors, diodes, fiber optic cables, and so forth). Some measurements are for a specific implementation of the device, and other implementations can have different values, such as certain dimensions made larger for a larger-sized product, or smaller for a smaller-sized product. The device may be made proportionally larger or smaller by adjusting relative measurements proportionally (e.g., maintaining the same or about the same ratio between different measurements). In various implementations, the values (or numbers or quantities) can be the same as the value given, about the same of the value given, at least or greater than the value given, or can be at most or less than the value given, or any combination of these. The values (or numbers or quantities) can also be within a range of any two values given or a range including the two values given. When a range is given, the range can also include any number within that range to any other number within that range.

The dimensions, for example, along an axis, a rotational orientation, or both are approximate values. The dimensions can be in values, directions, angles, or any combination of these dimension. Dimensions, for example, of values in millimeters or centimeters, of directions along an axis or at an angular orientation relative to an axis, of an angular orientation are approximate values. The values, direction, and angles can vary due to, for example, measurement or manufacturing tolerances or other factors. A dimension can vary, for example, by plus or minus 0.1 percent, plus or minus 0.2 percent, plus or minus 0.5 percent, plus or minus 1 percent, plus or minus 5 percent, plus or minus 10 percent, plus or minus 15 percent, plus or minus 20 percent, plus or minus 0.1 to 0.2 percent, plus or minus 0.2 to 0.5 percent, plus or minus 0.5 to 1 percent, plus or minus 1 to 5 percent, plus or minus 5 to 10 percent, plus or minus 10 to 15 percent, or plus or minus 15 to 20 percent.

The shapes, for example, a geometric shape can be approximate shapes. The shapes can be in values, directions, angles, terms, or any combination of these shapes. The shapes can vary due to, for example, measurement or manufacturing tolerances or other factors. A shape can vary, for example, by plus or minus 0.1 percent, plus or minus 0.2 percent, plus or minus 0.5 percent, plus or minus 1 percent, plus or minus 5 percent, plus or minus 10 percent, plus or minus 15 percent, plus or minus 20 percent, plus or minus 0.1 to 0.2 percent, plus or minus 0.2 to 0.5 percent, plus or minus 0.5 to 1 percent, plus or minus 1 to 5 percent, plus or minus 5 to 10 percent, plus or minus 10 to 15 percent, or plus or minus 15 to 20 percent.

The orientations, for example, parallel, perpendicular, transverse, and angle are approximate values. The orientation can be in values, directions, angles, terms, or any combination of these orientations. Orientations, for example, of terms or angles can be approximate orientations. The orientations vary due to, for example, measurement or manufacturing tolerances or other factors. An orientation can vary, for example, by plus or minus 0.1 percent, plus or minus 0.2 percent, plus or minus 0.5 percent, plus or minus 1 percent, plus or minus 5 percent, plus or minus 10 percent, plus or minus 15 percent, or plus or minus 20 percent. Terms, such as about, substantially, approximately, or other relative terms can include the described ranges as will be readily understood by those of ordinary skill in the art and can include ranges that will be understood by those of ordinary skill in the art.

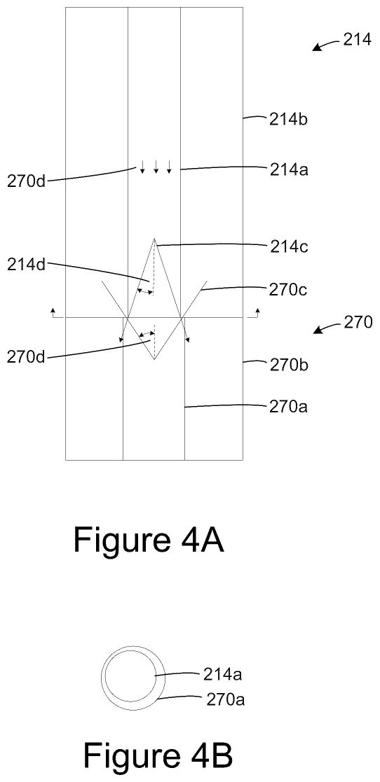

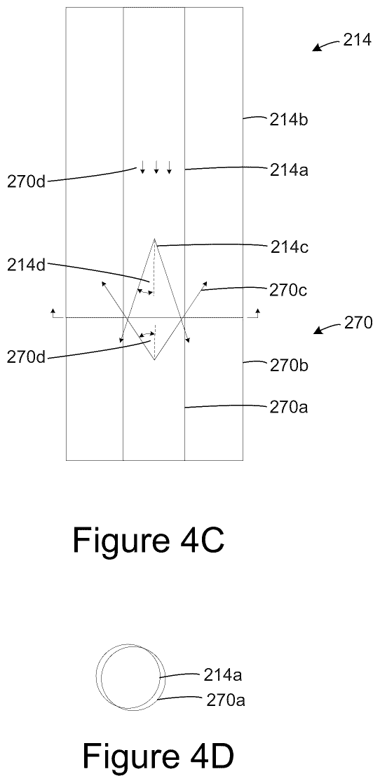

FIG. 4A shows the cores 214a and 270a of the source waveguides and shows the claddings 214b and 270b of these waveguides, in an implementation. The source waveguides are connected tip-to-tip when the probe unit is connected to the laparoscopic oximeter and more specifically when the bottom surface 305a of the baffle cap is connected to the top surface 20e of optical coupler 20d. When the tips of the core 214a of source waveguide 214 and the core 270a of source waveguide 270 are in contact, light can be transmitted from waveguide 214 into waveguide 270.

The downward arrows 270d in FIG. 4A show the direction of the travel of light from one of the source waveguides 214 to one of the source waveguides 270. More specifically, light generated and emitted from one of the light engines can be transmitted into source waveguides 214, and thereafter, the light can be transmitted from source waveguides 214 into source waveguides 270.

The core 214a of each source waveguide 214 has a diameter and numerical aperture that are respectively less than the diameter and numerical aperture of the core 270a of each source waveguide 270. The diameters and the numerical apertures of the cores of each of the source waveguides 214 can be equal. The diameters and the numerical apertures of the cores of each of the source waveguides 270 can also be equal. The diameter and the numerical aperture of the cores of each of the source waveguides 214 can be less than the diameters and the numerical apertures of each of the source waveguides 270.

More specifically, each source waveguide 214 has an emission cone 214c into which the waveguide can emit light. Each emission cone 214c has an emission angle 214d. The emission angle of a waveguide is the maximum transmission angle of light that is emitted from a waveguide. The emission angle is measured from a normal of the end of the waveguide to the maximum emission angle. The maximum emission angle can vary, e.g., increase or decrease, when the optical fiber emits light into different media, such as a liquid or air.

Each source waveguide 270 has an acceptance cone 270c. Each acceptance cone has an acceptance angle 270d. The acceptance angle is measured from a normal at the end of the waveguide to the maximum incidence angle at which light is transmitted into the waveguide. The maximum incidence angle can vary, e.g., increase or decrease, when the optical fiber emits light into different media, such as a liquid or air. Light received in the acceptance cone and at an angle that is less than or equal to the acceptance angle is transmitted into the fiber for transmission through the fiber. Light received outside of the acceptance cone and at an angle that is greater than the acceptance angle is either not transmitted into the waveguide or has a low probability of being transmitted into the waveguide. The numerical aperture is the sine of the emission angle or the acceptance angle and is a dimensionless number that characterizes the range of angles over which a waveguide can emit or accept light.

Emission angle 214d is less than acceptance angle 270d, in an implementation. Emission angle 214d is less than acceptance angle 270d by 1-30 degrees. Providing for emission angle 214d to be less than the acceptance angle 270d allows for a relatively high percentage (e.g., up to 96 percent) of light emitted from waveguide 214 to be accepted by waveguide 270. The light that does not enter waveguide 270 may be reflected back from the tip of source waveguide 270 towards the tip of source waveguide 214. Multiple reflections may result in a relatively constant noise signal in the laparoscopic oximeter.

The differences between the diameters and the numerical apertures of the cores of the source waveguides 214 and 270, as described above, allow for the source waveguides to be tip-to-tip connected but have the centers of the fibers misaligned while the percentage of light transmitted from the source waveguide 214 and received by the source waveguide 270 is the same for the configuration where the centers of the cores of the fibers are aligned. Thus, the mechanical couplers and pins that couple the probe unit and align the source waveguide can introduce small misalignments and the transmission of light can remain relatively high, such as remaining at about 96 percent.

FIG. 4B shows a cross-sectional view of the cores of source waveguides 214 and 270, where the cores are tip-to-tip connected, in an implementation. FIG. 4B also shows that the centers of the cores of the waveguides are misaligned, but the perimeter of the end of core 214a is within the perimeter of the end of core 270a. With the diameter and numerical aperture of core 214a less than the diameter and numerical aperture of core 270a, the transmission of light can remain relatively high, such as remaining at about 96 percent, when the centers of these waveguides are not aligned (i.e., not concentric). That is, an amount of tolerance (or slop) is permitted when the laparoscopic tube is connected to the probe unit to form the laparoscopic oximeter so that the laparoscopic oximeter can make reliable oximetry measurements.

FIG. 4C shows the cores 214a and 270a of the source waveguides and shows the claddings 214b and 270b of these waveguides. The source waveguides are connected tip-to-tip when the probe unit is connected to the laparoscopic oximeter and more specifically when the bottom surface 305a of the baffle cap is connected to the top surface 20e of optical coupler 20d. When the tips of the core 214a of source waveguide 214 and the core 270a of source waveguide 270 are in contact, light can be transmitted from waveguide 214 into waveguide 270.

The downward arrows 270d in FIG. 4C show the direction of the travel of light from one of the source waveguides 214 to one of the source waveguides 270. More specifically, light generated and emitted from one of the light engines can be transmitted into source waveguides 214, and thereafter, the light can be transmitted from source waveguides 214 into source waveguides 270.

The core 214a of each source waveguide 214 has a diameter that is substantially equal to the diameter of the core 270a of each source waveguide 270. The numerical aperture of the cores of each of the source waveguides 214 is less than the numerical aperture of the cores of each of the source waveguides 270. The cladding of each source waveguide 214 and the cladding of each source waveguide 270 can have different refractive indices such that the numerical apertures will be different as described.

More specifically, each source waveguide 214 has an emission cone 214c into which the waveguide can emit light. Each emission cone 214c has an emission angle 214d. The emission angle of a waveguide is the maximum transmission angle of light that is emitted from a waveguide. The emission angle is measured from a normal of the end of the waveguide to the maximum emission angle. The maximum emission angle can vary, e.g., increase or decrease, when the optical fiber emits light into different media, such as a liquid or air.

Each source waveguide 270 has an acceptance cone 270c. Each acceptance cone has an acceptance angle 270d. The acceptance angle is measured from a normal at the end of the waveguide to the maximum incidence angle at which light is transmitted into the waveguide. The maximum incidence angle can vary, e.g., increase or decrease, when the optical fiber emits light into different media, such as a liquid or air. Light received in the acceptance cone and at an angle that is less than or equal to the acceptance angle is transmitted into the fiber for transmission through the fiber. Light received outside of the acceptance cone and at an angle that is greater than the acceptance angle is either not transmitted into the waveguide or has a low probability of being transmitted into the waveguide. The numerical aperture is the sine of the emission angle or the acceptance angle and is a dimensionless number that characterizes the range of angles over which a waveguide can emit or accept light.

Emission angle 214d is less than acceptance angle 270d, in an implementation. Emission angle 214d is less than acceptance angle 270d by 1-30 degrees. Providing for emission angle 214d to be less than the acceptance angle 270d allows for a relatively high percentage (e.g., up to 96 percent) of light emitted from waveguide 214 to be accepted by waveguide 270. The light that does not enter waveguide 270 may be reflected back from the tip of source waveguide 270 towards the tip of source waveguide 214. Multiple reflections may result in a relatively constant noise signal in the laparoscopic oximeter.

The differences between the numerical apertures of the cores of the source waveguides 214 and 270, as described above, allow for the source waveguides to be tip-to-tip connected but have the centers of the fibers misaligned while the percentage of light transmitted from the source waveguide 214 and received by the source waveguide 270 is the same for the configuration where the centers of the cores of the fibers are aligned. Thus, the mechanical couplers and pins that couple the probe unit and align the source waveguide can introduce small misalignments and the transmission of light can remain relatively high, such as remaining at about 96 percent.

FIG. 4D shows a cross-sectional view of the cores of source waveguides 214 and 270, where the cores are tip-to-tip connected and are misaligned. With the numerical aperture of core 214a less than the numerical aperture of core 270a, the transmission of light can remain relatively high, such as remaining at about 96 percent, while the centers of the cores of these waveguides are not aligned (i.e., not concentric). That is, an amount of tolerance (or slop) is permitted when the laparoscopic tube is connected to the probe unit to form the laparoscopic oximeter so that the laparoscopic oximeter can make reliable oximetry measurements.

In an implementation, an optical element is positioned between the ends of optical fibers 214 and 270. The optical element can contact the ends of the optical fibers. The optical element can be a lens, a flat disk with substantially parallel sides that can contact the ends of the optical fibers, an elastic element that deforms to make contact with the ends of the optical fibers, or another element. The lens or flat element can be glass, plastic, or a plastic type material. The index of refraction of the flat element can match the refractive indices of one or both of the cores of the optical fibers.

In an implementation, the laparoscopic tube is calibrated and the calibration information for the laparoscopic tube is stored in communication tag 7. Calibration information for the laparoscopic tube can be generated by measuring a percentage of the transmission of light for a known amount of light transmitted into each waveguide 270, by measuring a percentage of the transmission of light for a known amount of light transmitted into each detector waveguide 275, or both.

When the laparoscopic tube is connected to the probe unit to form the laparoscopic oximeter 5, the tag reader 8 of the probe unit can access and read the calibration information stored in the communication tag 7. The calibration information collected by the tag reader can be used by the processor to calibrate the laparoscopic tube. More specifically, the calibration information retrieved from the communication tag 7 can be stored in memory 222 and retrieved and used by the processor before or when oximetry measurements are made to apply calibration adjustments to the measured data. Further, when different laparoscopic tubes are attached to the probe unit, calibration information specific to each laparoscopic unit that is generated during the manufacture of the laparoscopic tube can be collected by the tag reader and can be used by the processor to uniquely calibrate each laparoscopic tube and measurement data collected by the laparoscopic tube.

Other devices and transmission protocols can be used to transmit calibration information for the laparoscopic tube to the probe unit. For example, calibration information can be supplied to the probe unit via a computer that stores the calibration information. The transmission protocol may be an Ethernet protocol, a USB protocol, a UART protocol (i.e., universal asynchronous receiver transmitter protocol), a USART protocol (i.e., a universal synchronous asynchronous receiver transmitter protocol), an SPI Protocol (i.e., serial peripheral interface bus protocol), or another wired communication protocol.

A wireless communication link can also be used to transmit the calibration information from a computer to the probe unit. The wireless communication link can operate according to one of a variety of protocols, such as one of the Bluetooth protocols (e.g., Bluetooth, Bluetooth SMART, Bluetooth Low Energy, others), one of the IEEE 802.11 protocols, ANT, 6LoWPAN, MyriaNed, EnOcean, Z-Wave, Wi-Fi, one of the IEEE 802.15.4 protocol, such as ZigBee, or others. These or other wireless protocols can be used by the probe unit to receive and transfer data to and from a computer system.

The wireless link between the laparoscopic oximeter and the computer system is a direct wireless connection in an implementation. That is, no intermediary transmitter circuits, receiver circuits, or transceiver circuits receive the wireless signal transmitted from the computer system to the probe unit for subsequent retransmission of the wireless signal to the probe unit. Similarly, no intermediary transmitter circuits, receiver circuits, or transceiver circuits receive the wireless signal transmitted from the probe unit to the computer system for subsequent retransmission of the wireless signal to the computer system.

In an implementation, the probe unit includes a user interface via which a user can enter the calibration information for a laparoscopic element. The user interface can be a user interface of the display element 205 that can include a touch screen adapted to receive the user input for the calibration information. The probe unit can include other user input devices, such as one or more buttons or a keypad, adapted for receiving the calibration information.

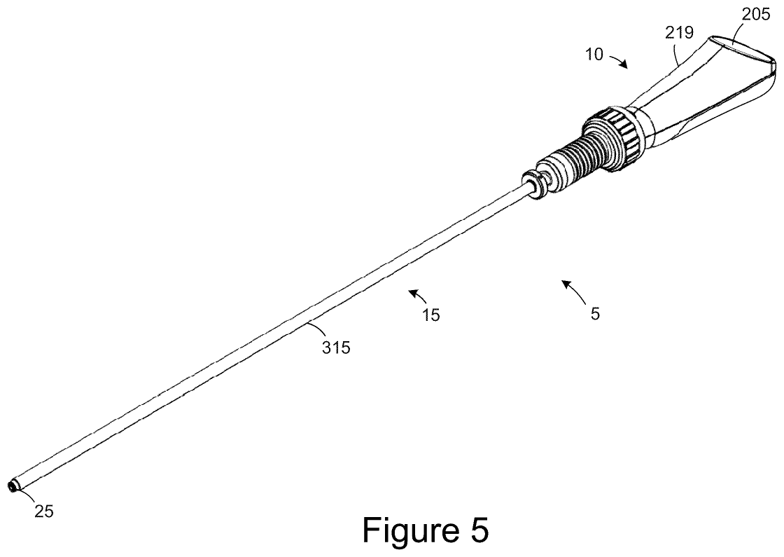

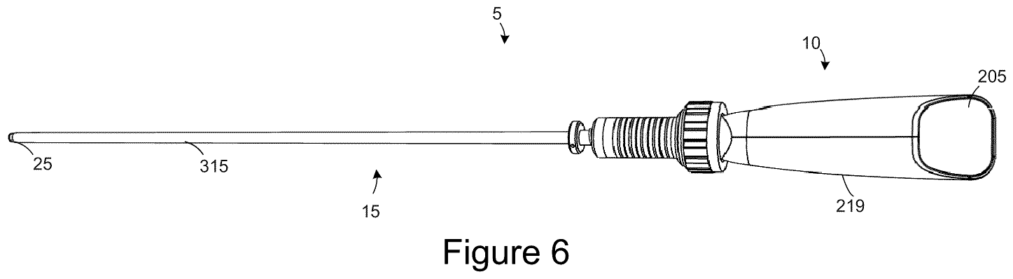

FIGS. 5-6, respectively, show a perspective view and a planar view of laparoscopic oximeter 5, in an implementation. The figures show the probe unit removably connected to the laparoscopic tube. The sensor head and display are located at opposite ends of the laparoscopic oximeter. Thus, when the extended tube 315 of the laparoscopic tube is positioned for surgery inside a patient, the display is readably visible to an operator operating the laparoscopic tube. The figures show the sensor head 25 extending from elongated tube 315.

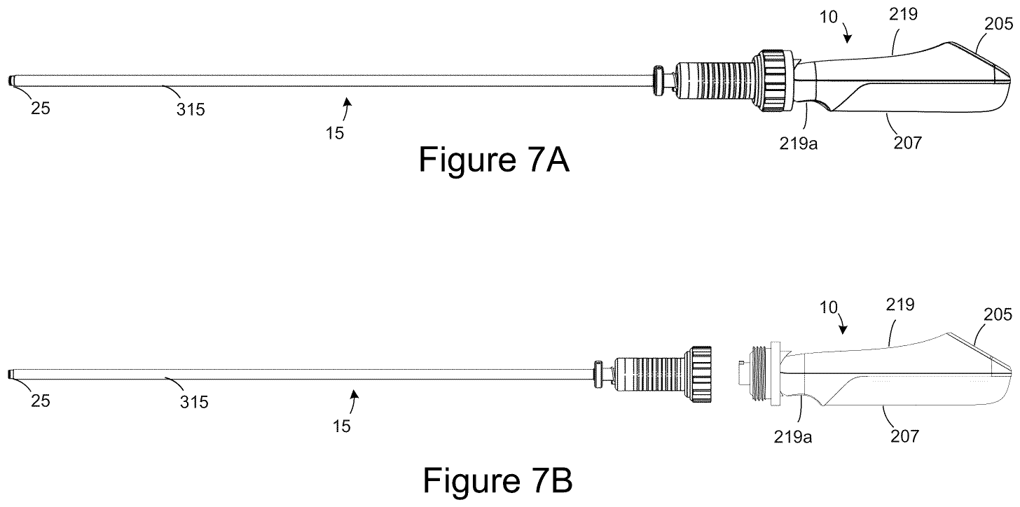

FIGS. 7A-7B show side views of the laparoscopic oximeter with a battery housing 207 of the probe unit. FIG. 7A shows the probe unit and laparoscopic tube connected and FIG. 7B shows the probe unit and laparoscopic tube disconnected. The battery housing can be detachable from the probe unit so that the battery housing and batteries housed by the battery housing can be replaced when the batteries become sufficiently discharged such that the battery may not power the probe unit through a surgery.

In the side-planar view, the face of the display is shown at an acute angle with respect to the long axis (the left to right direction of the plane of the page of the figures) of the laparoscopic tube. In some implementations, The display can be angled from 0 degrees to 80 degrees from the long axis of the laparoscopic tube so that the display can point towards an operator's face when the laparoscopic tube is inserted into a patient. Various embodiments of the laparoscopic oximeter can have different angular orientations of the display and long axis of the laparoscopic tube.

The laparoscopic oximeter is fully self-contained and does not have to connect to another device to be operational for a surgery. The laparoscopic oximeter can make the oximetry measurements described above and display the oximetry measurements, such as oxygen saturation values of tissue, based on the oximetry measurements without connecting to another device.

Housing 219 of the probe unit of the laparoscopic oximeter is a handheld housing that can be held by an operator in their hand, such as when the laparoscopic oximeter is used during a surgery or calibration. In a specific implementation of the handheld housing, the housing includes a finger rest 219a (e.g., a concave indentation in the handheld housing) located near an end of the probe unit that connects to the laparoscopic tube. The finger rest can be positioned between the battery housing and the end of the probe unit. The finger rest aids the handheld use of the laparoscopic oximeter so that the handheld housing does not slide forward in a user's hand. In other implementations of the handheld housing, the housing does not include the finger rest shown in FIGS. 7A-7B but has a relatively straight profile. The handheld housing is an elongated rectangular block shape with rounded sides so that the handheld housing is comfortable to hold for extended periods of time (e.g., one or more hours) during a surgery. The handheld housing adjacent to the display can flare so that the handheld housing can be held comfortably in a user's hand and so that the laparoscopic oximeter does not slip forward in a user's hand during use.

When the laparoscopic tube is disconnected from the probe unit, such as after a surgery, the laparoscopic tube can be discarded and the probe unit can be reused. To reuse the probe unit, a different laparoscopic tube can be connected to the probe unit so that another surgery can be performed.

FIGS. 8-9, respectively, show a perspective view of probe unit 10 and a perspective view of laparoscopic tube 15, in an implementation. Probe unit 10 includes a threaded sleeve 800 that fits around a side surface of baffle cap 305. The threaded sleeve is positioned at an end of the probe unit that is at an opposite end at which the display is located. Laparoscopic tube 15 includes a threaded locking cap 900 that is located at an end of the laparoscopic tube that is at an opposite end of the laparoscopic tube where sensor head 25 is located.

Threaded sleeve 800 includes a set of threads 805 and threaded locking cap 900 includes a set of threads 905. Threaded sleeve 800 includes outward-facing threads and threaded locking cap 900 includes inward-facing threads. In an alternative implementation, threaded sleeve 800 includes inward-facing threads and the threaded locking cap includes outward-facing threads. The threads of the threaded sleeve can be threaded into the threads of the threaded locking cap to releasably connect the probe unit to the laparoscopic tube. In an implementation, the threaded sleeve is configured to rotate with respect to the baffle cap so that the baffle cap does not rotate when the threaded sleeve is rotated.

The baffle cap includes a tab 810 that extends from sidewall 305b of the baffle cap. The tab is configured to operate as part of a key system, which includes a slot formed in one or more components of the laparoscopic tube. A slot of the key system is described below. The key system facilitates the alignment of the tips of source optical fibers 214 to the tips of the source optical fibers 270 and facilitates the alignment of the detector optical fibers 275 to the photodetectors 335.

The perspective view of the probe unit shows the photodetectors 335 fitted into the apertures that are formed in the end wall of the baffle cap. The top surfaces of the detectors can be flush with the bottom surface 305a of the baffle cap. The perspective view of the probe unit also shows the apertures in which the ends of source optical fibers 214 are located.

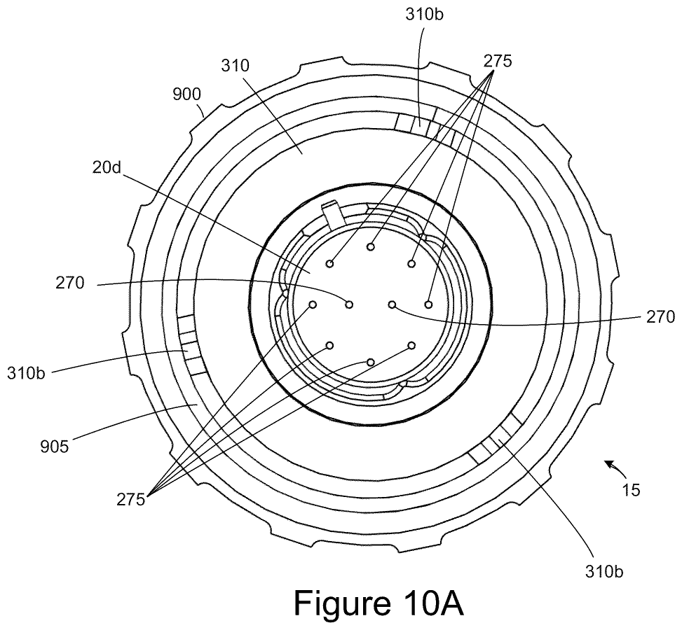

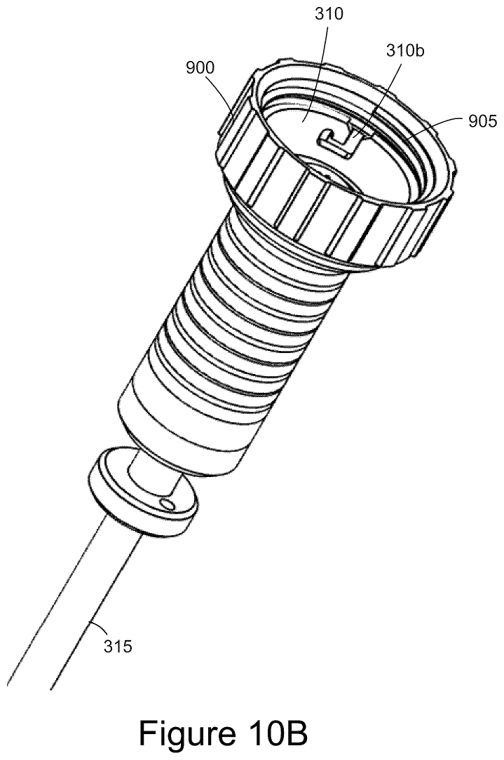

FIGS. 10A-10B show end views of laparoscopic tube 15. FIG. 10A shows a planar view of the end of the laparoscopic tube where the laparoscopic tube connects to the probe unit. FIG. 10B shows a perspective view of the end of the laparoscopic tube where the laparoscopic tube connects to the probe unit.

As shown in FIG. 10A, the apertures formed in optical coupler 20d for detector optical fibers 275 are arranged in a circular configuration. The apertures can be arranged in other configurations, such as oval, square, rectangular, two arcs with four apertures per arc where the arcs do not form a circular configuration of apertures, or another shape. The apertures formed in optical coupler 20d for source optical fibers 270 are located inside the circle of the circular configuration of the detector optical fibers. In an embodiment, the two apertures for the source optical fibers are in line with the two apertures for the detector optical fibers. The tips of the source optical fibers are flush with the top surface of the aperture plate shown in FIG. 10A. The tips of the detector optical fibers can also be flush with the top surface of the aperture plate, in an implementation.

Slots 310b are arranged in a triangular configuration, such as an equal lateral triangle configuration. Threads 905 encircle the slots. FIG. 10B shows one of slots 310b and shows the “J” shape of the slot. Each slot can similarly have the “J” shape for receiving pins 305d.

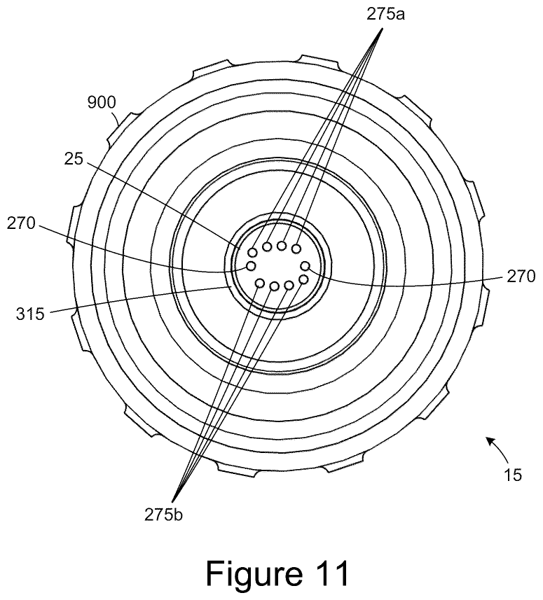

FIG. 11 shows an end view of the laparoscopic tube where sensor head 25 is located in elongated tube 315. FIG. 11 also shows an end view of threaded locking cap 900, where the threaded locking cap has a greater diameter than the diameter of elongated tube 315 and is visible from the end view of the laparoscopic tube.

The apertures formed in sensor head 25 for detector optical fibers 275 are arranged in two arcs where the first arc includes apertures for detector optical fibers 275a and the second arc includes apertures for detector optical fibers 275b. The apertures can be arranged in other configurations, such as circular, oval, square, rectangular, or another shape. The two arcs can have the same shapes. Corresponding apertures on the arcs are symmetric about a point between the two arcs, such as a point (e.g., a center point) on the line connecting the two apertures for the source optical fibers, in an implementation. The apertures formed in sensor head 25 for source optical fibers 270 are arranged on a line that is between the two arcs of apertures for the detector optical fibers. The apertures for the source fibers can be the most distantly spaced apertures in the sensor head.

The arrangement of apertures for the detector optical fibers in optical coupler 20d and sensor head 25 are different arrangements of apertures, in an implementation. For example, the arrangement of the apertures and the locations of the tips of the detector optical fibers in the sensor head facilitates the use of spatially resolved tissue oximetry by the probe unit for determining oximetry measurements for tissue. The arrangement of apertures and the tips of the detector optical fibers in the optical coupler are separated around a circle near the side of the optical coupler to lower cross contamination of light transmitted from the detector optical fibers to the photodetectors. Thus, accurate oximetry measurements can be made by the laparoscopic oximeter because light cross-talk is lowered at the photodetectors.

The apertures in the sensor head for the source optical fibers, the tips of the source optical fibers, waveguides (e.g., an epoxy coating over the tips of the source optical fibers) that optically couple to the source optical fibers, or any combination of these elements are sometimes referred to as source structures. The apertures in the sensor head for the detector optical fibers, the tips of the detector optical fibers, waveguides (e.g., an epoxy coating over the tips of the detector optical fibers) that optically couple to the detector optical fibers, or any combination of these elements are sometimes referred to as detector structures.

While the sensor head is shown in FIG. 11 to include eight detector structures and two source structures, the sensor head can include more or fewer source structures and more or fewer detector structures. The source structures emit light from optical fibers 270 of the laparoscopic tube and the detector structures detect light reflected from tissue, such as patient tissue or a tissue proxy. The detector structures can transmit light to detector optical fibers 275 of the laparoscopic tube.

In an implementation, spatially resolved spectroscopy is facilitated by the source structure to detector structure distances of the sensor head where at least one source structure to detector structure distance is less than 1.5 millimeters and can be less than one millimeter, and at least one source structure to detector structure distance is greater than 2.5 millimeters or at least two source structure to detector structure distances are greater than 2.5 millimeters. However, the detector structures can have other spacing between them, for example, based on the particular tissue that the laparoscopic oximeter is used for, such as the colon, tissue supporting the colon (such as the mesentery tissue), arteries supplying blood to the colon, or other tissue. The source structure to detector structure distances can be achieved via a number of source and detector combinations, such as one source and two detectors, two sources and one detector, or other combinations.

Spatially resolved spectroscopy is further facilitated by the memory storing and the processor using a number of simulated reflection curves, where each reflection curves represent an absorption coefficient and a scattering coefficient for the particular configuration of source structures and detector structures of the sensor head.

The simulated reflection curves include reflection intensities (e.g., in arbitrary units) for light reflected from simulated tissue for a variety of wavelengths emitted from the laparoscopic oximeter. The simulated reflectance curves can be of simulated tissue using a Monte Carlo simulation method. The processor can determine one or more of the simulated reflectance curves that best fits (e.g., lowest fit error determined by a fit method, such as least squared or others) reflectance data generated by the detectors. The processor can then determine one or more absorption coefficients and one or more scattering coefficients for the tissue from the one or more simulated reflectance curves that best fits the reflectance data. From the absorption coefficient, the processor can then determine other oximeter information for measured tissue, such as oxygen saturation. The source structure to detector structure distances of the sensor head facilitate that the absorption coefficient and the reduced scattering coefficient can be determined from the simulated reflectance curves where these coefficients are mathematically independent. Because the absorption coefficient and the reduced scattering coefficient are mathematically independent, further tissue measurements, further mathematical determinations, or both can be avoided, via the use of such spatially resolved spectroscopy to determine oximetry information for measured tissue.

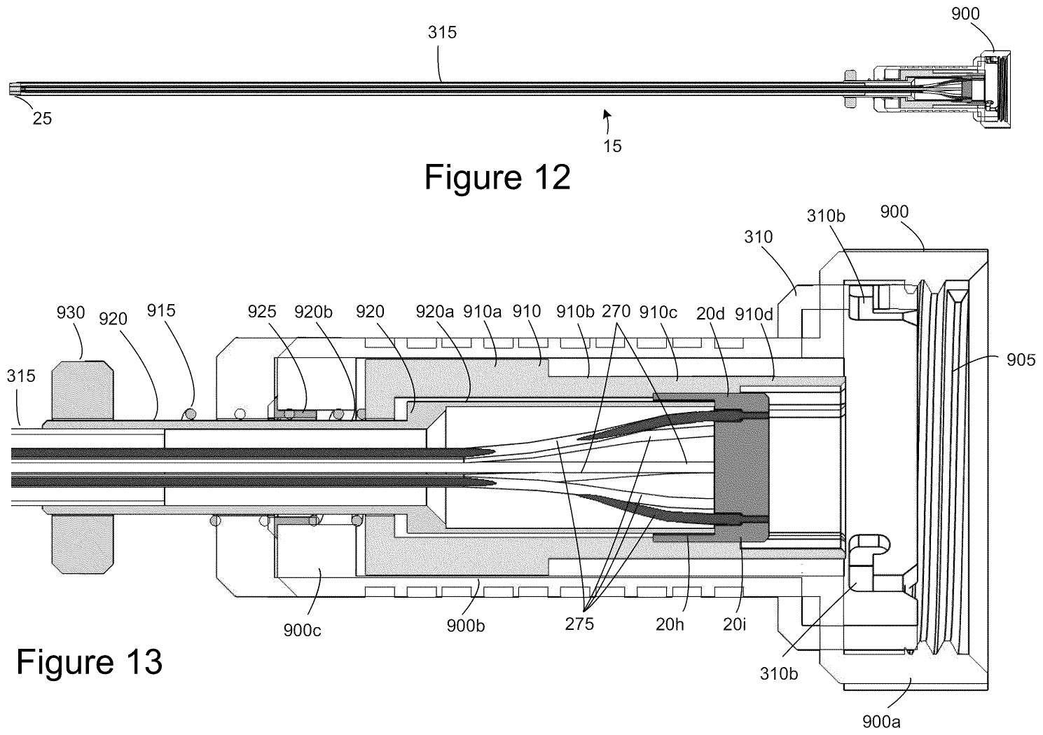

FIGS. 12-13 show cross-sectional views of laparoscopic tube 15, in an implementation. As shown in the cross-sectional views, threaded locking cap 900 houses the optical coupler 20d, a bearing 925, a collet 910, houses at least a portion of a spring 915, and a fiber tube adapter 920. A spring stop 930 is located around an end portion of the fiber tube adapter 920. Spring stop 930 can be connected to fiber tube adapter 920 via an adhesive (such as epoxy), a fastener (such as a screw, a grub screw (i.e., headless screw)), or both.

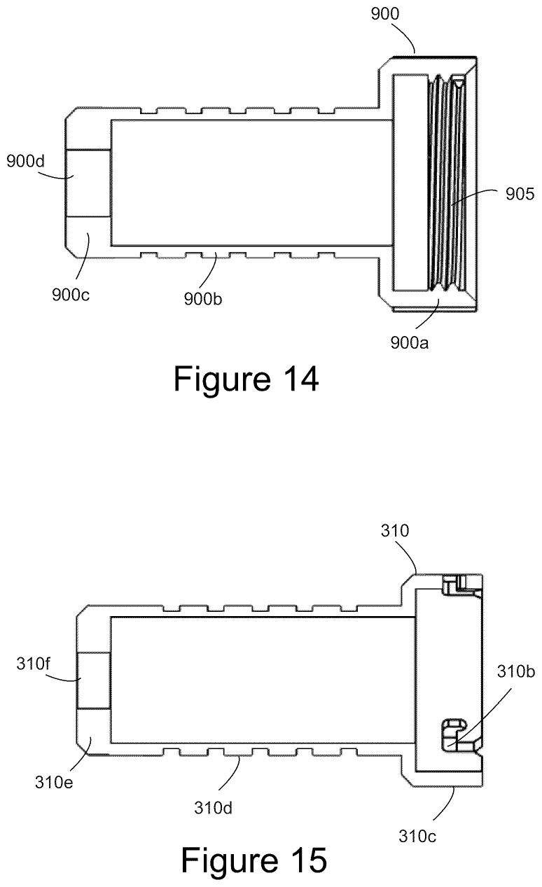

FIG. 14 shows a cross-sectional view of threaded locking cap 900. The threaded locking cap includes a cap 900a, a tube 900b, and an end cap 900c. Cap 900a is tube-shaped and includes internal threads 905. Tube 900b is connected to cap 900c and includes an open interior space. End cap 900c is positioned inside the interior space of tube 900. The end cap includes an aperture 900d formed in the cap. The outside diameter of cap 900a is greater than each of the outside and inside diameters of tube 900b. The inside diameter of cap 900a is greater than each of the outside and inside diameters of tube 900b. The inside surface of tube 900b is a smooth surface. The inside surface of the aperture of end cap 900c is a smooth surface. At least a portion of spring 915 is located in the aperture of the end cap.

FIG. 15 shows a cross-sectional view of housing 310 (sometimes referred to as a lockable spring-loaded cap). Threaded locking cap 900 can be integrally formed with housing 310 or can be separate elements of the laparoscopic tube. In an implementation, the threaded locking cap 900 and housing 310 are rotatable with respect to one another so that pins 305d can be in the J shaped portions of slots 310d of housing 310 and locking cap 900 can be rotated to thread couple to the threads of threaded sleeve 800.

Housing 310 includes a top 310c that includes slots 310b. Housing 310 also includes a tube 310d and an end cap 310e. End cap 310e includes an aperture 310f formed in the cap. The diameter of aperture 900d is greater than the diameter of aperture 310f. The diameter of end cap 310c is greater than each of the diameters of the tube 910d and end cap 310c.

Referring again to FIG. 13, bearing 925 is fitted into aperture 900d of threaded locking cap 900. Bearing 925 can be formed of a variety of materials with low static friction, such as polytetrafluoroethylene, nylon, polyester, or another plastic-type material.

Collet 910, sometimes referred to as a compression collet, is located in the interior space of the tube section 900b of the threaded locking cap. The collet includes a first tube 910a, a second tube 910b connected to the first tube, a third tube 910c connected to the second tube, and a fourth tube 910d connected to the third tube. An outside surface of first tube section 910a contacts an inside surface of tube section 900b of threaded locking cap 900. The outside surface of first tube section 910a is configured to be in sliding contact with the inside surface of tube section 900b of threaded locking cap 900. Thus, collet 900 can slide in the direction of the long axis of the threaded locking cap. In an implementation, collet 900 can also rotate with respect to the threaded locking cap.

Optical coupler 20d includes a tube 20h that is connected to a round plate 20i. The round plate includes the apertures that are described above in which the end portions of source optical fibers 270 and detector optical fibers 275 are located. The tube portion of the optical coupler has an opening that faces towards elongated tube 315.

Collet 910 includes a step in the interior of the collet where second tube section 910b meets third tube section 910c. At the step, the interior diameter of the second tube section is less than the diameter of the third tube section. The end of the tube section 20h of the optical coupler contacts the step at the second and third sections of the collet.

Fiber tube adapter 920 includes a first tube 920a that is connected to a second tube 920b where each of the inside and outside diameters of the first tube is greater than each of the inside and outside diameters of the second tube. An end of the first tube 920a is located in the opening formed by tube 20f of the optical coupler 20d. The end of the first tube 920a contacts the surface of the round plate 20i of the optical coupler.

A portion of the second tube 920b of the fiber tube adapter 920 is located in the central aperture of bearing 925. The second tube is adapted to slide inside the bearing. The second tube can slide along the length of the tube (left and right in FIG. 13), can rotate inside the bearing, or both.

The second tube 920b is also located inside a central opening of spring 915. A first end of spring 915 contacts an end of collet 910 and a second end of the spring contacts spring stop 930.

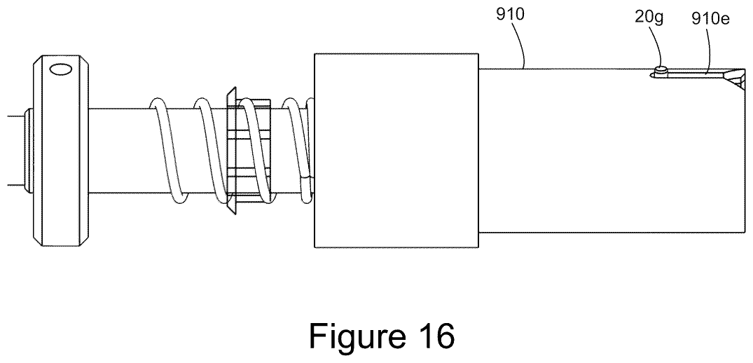

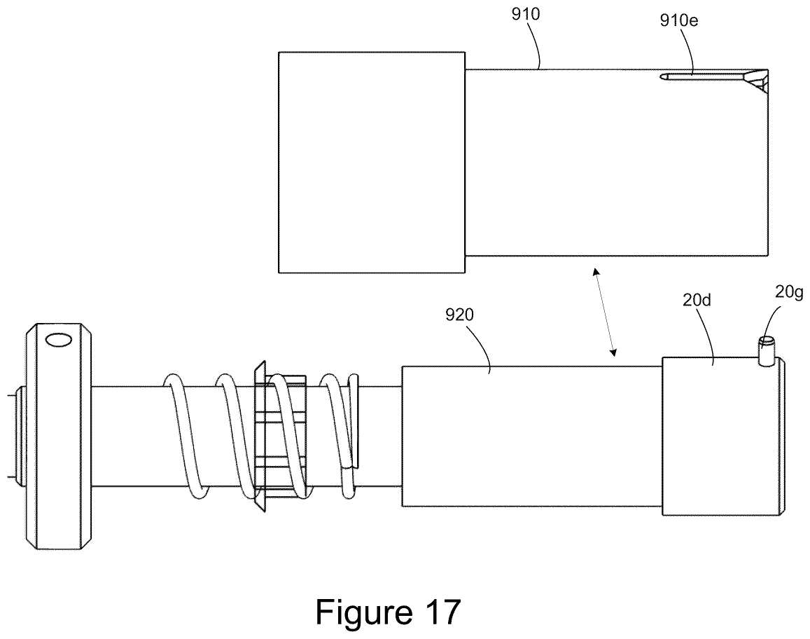

FIGS. 16-17 show the outside surface of collet 910. FIG. 17 also shows the outside surface of optical coupler 20d and fiber tube adapter 920. The collet includes a slot 910e that is located in the third tube 910c, the fourth tube 910d, one of these tubes, or both of these tubes. The slot can extend through the sidewall of the collet. The entrance of the slot at the end surface of the collet is beveled on both sides of the slot. The optical coupler includes a pin 20g that extends from an outer sidewall of the coupler.

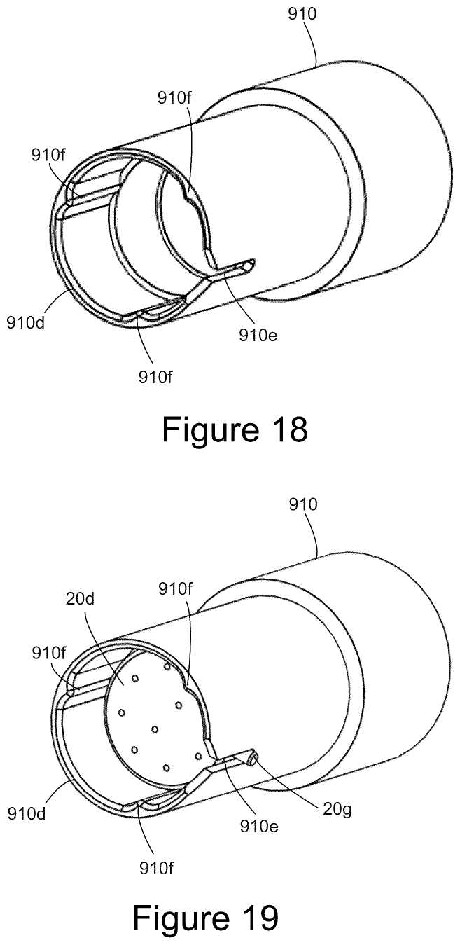

FIGS. 18-19 show perspective end views of collet 910, in an implementation. The collet includes a number of ridge protrusions 910f that extend from the inner sidewall of the fourth tube 910d of the collet. The number of ridge protrusions can include three or more ridge protrusions. The ridge protrusions extend from the end surface of the collet to the step that is located where the third and fourth tubes meet inside the collet. From an end view, the three ridge protrusions are arranged in an equilateral triangle arrangement or other triangular arrangement. In an implementation, the inner sidewall of the collet does not include ridge protrusion 910f but has a relatively smooth surface. For example, if the space between the outer surface of the optical coupler 20d and the inner sidewall of collet 910 is relatively small, as will be understood by those of ordinary skill in the art, the source optical fibers 214 will acceptably align with the source optical fibers 270 and the detector optical fibers 275 will acceptably align with the photodetectors 335.

FIG. 19 shows the optical coupler located inside the collet where pin 20g is located in slot 910e and the outside surface of the optical coupler 20d is in contact with the ridge projections 910f. During assembly of laparoscopic tube 15, optical coupler 20d is positioned inside collet 910, pin 20g is slid into slot 910e, and the outside surface of the optical coupler contacts with the tips of ridge protrusion 910f. The pin located in the slot sets the angular orientation of the optical coupler and the apertures formed in the optical coupler with the collet and the slot. The three ridge protrusions set the location of the central axis of the optical coupler relative to the central axis of the collet. In an implementation, the central axes of the optical coupler and the collet align when the optical coupler is located in the collet.

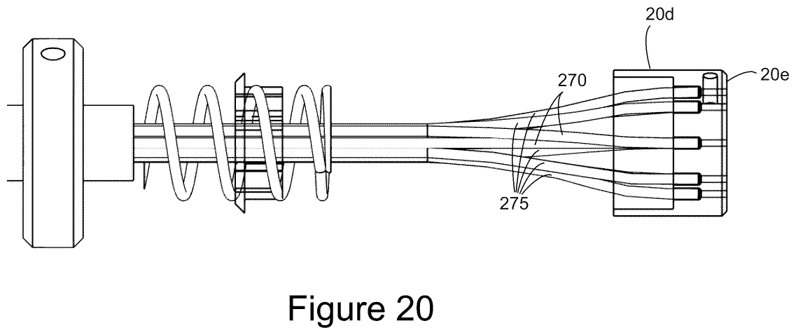

FIG. 20 shows a transparent view of optical coupler 20d, in an implementation. As shown in the figure, the ends of the source optical fibers 270 and the ends of the detector optical fibers 275 are located in the apertures formed in the optical coupler. The tips of the source optical fibers 270 and the tips of the detector optical fibers 275 are flush with the top surface 20e of the optical coupler.

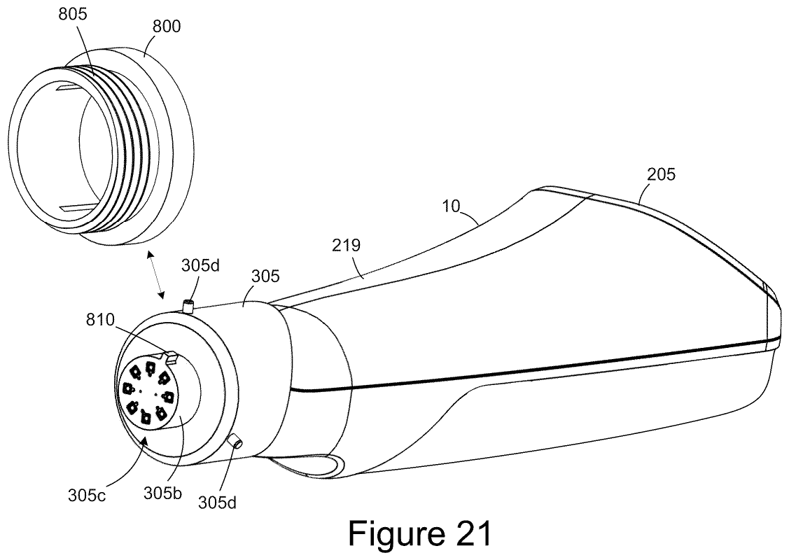

FIG. 21 shows a perspective view of the probe unit 10, in an implementation. The end view shows the probe unit with the threaded sleeve 800 removed from the probe unit. With the threaded sleeve shown removed from the probe unit, the baffle cap 305 is shown in perspective. Two of the three pins 305d are shown extending from the baffle cap. Tab 810 is also shown extending from the baffle cap. Tab 810 is adapted to slide into slot 910e of the collet when the probe unit is connected to the laparoscopic tube.

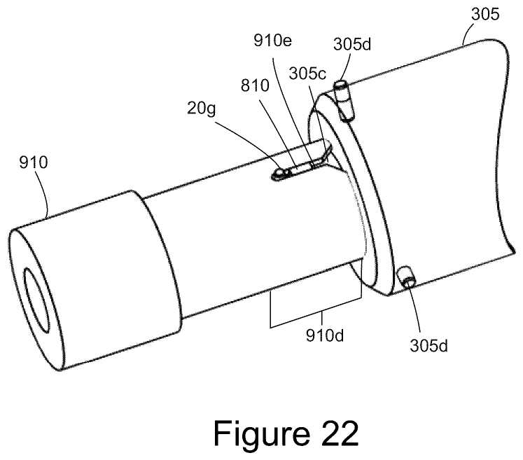

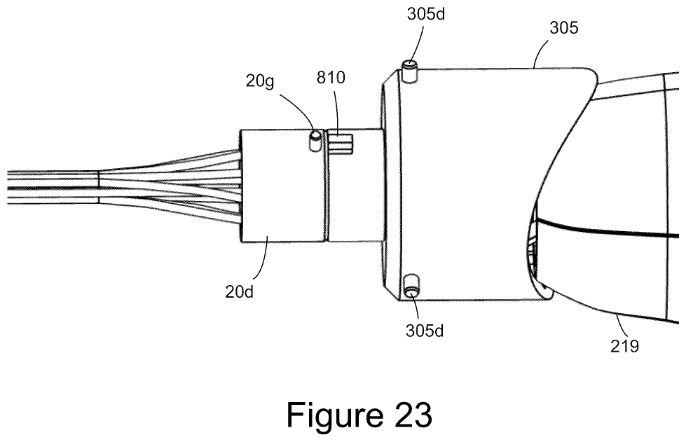

FIG. 22 shows baffle cap 305 attached to collet 910 and FIG. 23 shows the optical coupler butted against the baffle cap (the collet is not shown in FIG. 23). As shown in FIG. 22, an end portion 305c of the collet is positioned in the fourth tube 910d of the collet. Both pin 20g and tab 810 are poisoned in slot 910c. The sidewall 305b of end portion 305c contacts ridge protrusions 910f. Similar to the pin of the optical coupler located in the slot and the sidewall of the optical coupler in contact with the ridge protrusion, tab 810 located in the slot sets the angular orientation of the baffle and the apertures formed in the baffle cap with the collet and the slot. Additionally, when tab 810 is located in the slot, the tab and slot set the angular orientation of the apertures of the optical coupler with the apertures of the baffle cap. Additionally, when tab 810 is located in the slot, the tab and slot set the angular orientation of source optical fibers 214 to source optical fibers 270 and set the angular orientation of photodetectors 335 to detector optical fibers 275.

The three ridge protrusions set the location (i.e., distance) of the central axis of the baffle cap relative to the central axis of the collet. In an implementation, the central axes of the baffle cap and the collet align when the optical coupler is located in the collet. Thus, the slot, pin, tab, ridge protrusions, and side walls of the optical coupler of the baffle cap facilitate the optical coupling of source optical fibers 214 and 270 and the optical coupling of the detector optical fibers and the photodetectors.

More specifically, in an implementation, the probe unit and the elongated tube include a number of alignment systems to align the source optical fibers of the probe unit and the elongated tube. A first alignment system of the number of alignment systems includes slot 810, which is a key slot located in the collet and a pin 20g, which is a first key located on an optical coupler, where the first key is positioned in the key slot sets a rotational orientation of the collet with respect to the optical coupler and a first number of optical fibers that include ends of the optical fibers housed by the optical coupler.

A second alignment system of the number of alignment systems includes the key slot and tab 810, which is a second key located on the baffle cap of the probe unit, where the second key positioned in the key slot sets a rotational orientation of the collet, the optical coupler, and the first optical fibers with respect to the probe unit, second optical fibers of the probe unit, and photodetectors of the probe unit.

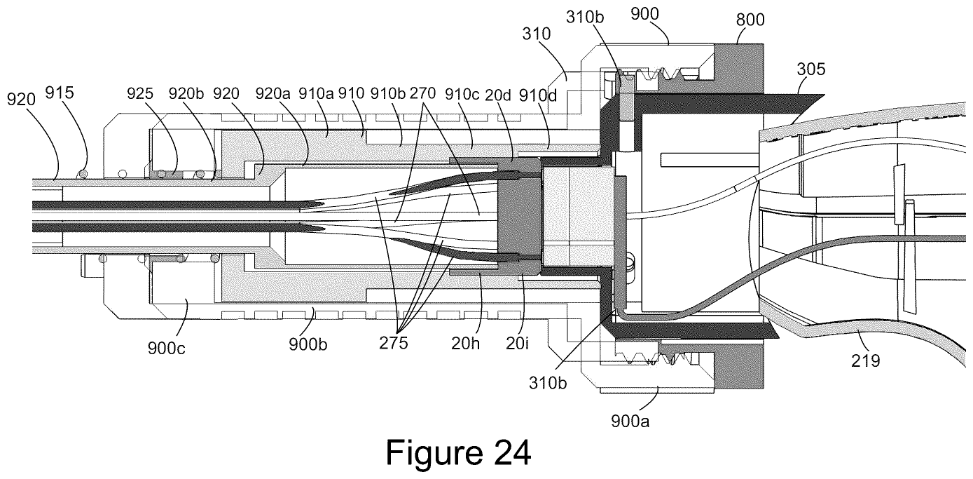

A third alignment system of the number of alignment systems includes ridges 910f formed on an inside surface of the tubular portion of the collet and includes the side surface (i.e., outer surface) of the optical coupler. When the ridges contact the side surface of the optical coupler, a first distance is set between a central axis of the collet to a central axis of the optical coupler. The central axis of the collet extends from left to right with respect to the plane of the page of FIG. 24 and passes through the center of the collet, which is sometimes referred to as a longitudinal axis of the collet. The central axis of the optical coupler extends from left to right with respect to the plane of the page of FIG. 24 and passes through the center of the optical coupler, which is sometimes referred to as the longitudinal axis of the optical coupler.

A fourth alignment system of the number of alignment systems includes ridges 910f formed on an inside surface of the tubular portion of the collet and an outer surface of the baffle cap of the probe unit. When the ridges contact the outer surface of the baffle cap of the probe unit, a second distance is set between the central axis of the collet to a central axis of the baffle cap of the probe unit. The central axis of the baffle cap extends from left to right with respect to the plane of the page of FIG. 24 and passes through the center of the baffle cap, which is sometimes referred to as the longitudinal axis of the baffle cap.

A distance between centers of connected tips of source optical fibers 214 and tips of source optical fibers 270 is based on the first and second distances between the described axes. In an implementation, the distance between the centers of the connected tip is a difference between the first and second distances. The distance between the centers of the connected tips is less than a radius of each second optical fiber.

A distance between each of the optically coupled centers of the tips of source optical fibers 270 and the centers of photodetectors 335 is based on the first and second distances between the described axes. In an implementation, the distance between each of the optically coupled centers of the tips of source optical fibers 270 and the centers of photodetectors 335 is a difference between the first and second distances.

FIG. 24 shows a cross-sectional view of probe unit 10 coupled to laparoscopic tube 15, in an implementation. When baffle cap 305 is moved into contact with the threaded locking cap 900 and housing 310 to connect the probe unit to the laparoscopic tube, pins 305d slide in slots 310b. Additionally, when baffle cap 305 is pressed into contact with optical coupler 20d, the optical coupler presses on both collet 910 and fiber tube adapter 920. The optical coupler 20d, collet 910, and fiber tube adapter 920 move longitudinally within threaded locking cap 900. That is, the optical coupler, collet, and fiber tube adapter move along the central axis of the threaded locking cap away from the probe unit and toward the sensor head of the laparoscopic tube (left in FIG. 24).

Spring 915 is positioned to contact an end of the collet 910. The spring compresses when the collet and the fiber tube adapter are pressed on by the baffle cap and when the collet and fiber tube adapter move longitudinally in the threaded locking cap 900. The spring applies a restoring force on the optical coupler, collet, and fiber tube adapter. The baffle cap can be rotated so that pins 305d are rotated and the pins can enter the J portions of slots 310b. When the pins are in a position to enter the J portions of the slots, the spring applies a spring force to push the pins into the slots.

When the baffle cap is rotated so that the pins can enter the J portions of slots 310b, one or more of the optical coupler, collet, and fiber tube adapter rotates so that the angular orientation of the optical coupler and baffle cap is not changed with respect to each other. Pin 20g and tab 810 located in slot 910e of the collet facilitate the rotation of the optical coupler and the collet when the baffle cap is rotated so that the pins enter the “J” portion of the slots. Additionally, the alignment of source optical fibers 214 and 270 is unchanged and the alignment of the detector optical fibers and the photodetector is unchanged. When pins 305d are positioned in the J portions of slots 310b, spring 915 continues to place a force on the collet so that the optical coupler is pressed into contact with the baffle cap so that a compression force is place on the tips of the source optical fibers that are coupled tip-to-tip. The spring constant of spring 915 is about 0.0565 newtons/meters (e.g., 0.5 pounds/inch) to about 0.565 newtons/meter (e.g., 5 pounds/inch). Spring 915 is compressed about 1.5 centimeters to about 2.5 centimeters (about 2 centimeters in one embodiment) when the spring is compressed when the laparoscopic tube is coupled to probe unit 10.

In an implementation, threaded cap 800 is configured to slide over baffle cap 305 and thread into the threads of the threaded locking cap 900. The threaded locking cap places an additional force that presses the baffle cap into the laparoscopic tube, and, more specifically, into the optical coupler, the collet, one of these elements, or both of these elements. The threaded locking cap further secures probe unit 10 to laparoscopic tube 15 to form the laparoscopic oximeter 5. In FIG. 24, the threaded cap is shown threaded into the threaded locking cap. The probe unit can be separated from the laparoscopic tube by first unthreading the threaded cap from the threaded locking cap, releasing the pins from the “J” slots, and pulling the probe unit and laparoscopic tube apart.

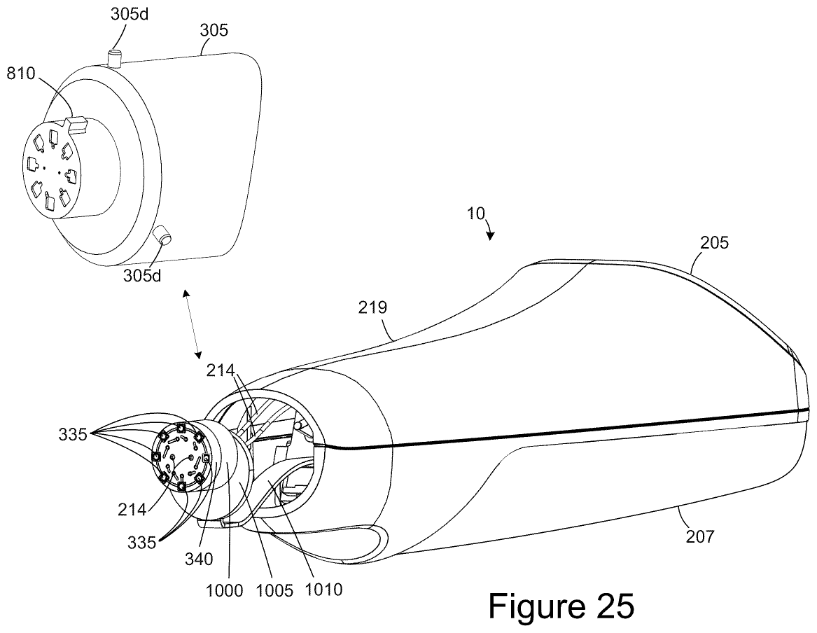

FIG. 25 shows a perspective view of the probe unit with the threaded cap and baffle cap removed from the probe unit, in an implementation. With the caps removed, first PCB 340 is revealed and shown, a second PCB 1000 is revealed and shown, and a third PCB 1005 is revealed and shown. As described above, photodetectors 335 are located on and attached to a front surface of the first PCB. The photodetector may be arranged in a circular configuration or another configuration. The first PCB includes first and second apertures in which the ends of source optical fibers 214 are located. The ends of the optical fibers can extend from the front surface of the first PCB so that the tips of the optical fibers are flush with the front surface of the baffle cap when the baffle cap is on the probe unit.

In an implementation, the first PCB includes electrical traces that extend from the front surface of the first PCB to a second surface of the first PCB to bring power and ground to the photodetectors and to allow for the electrical signals generated by the photodetectors to be transferred to the second side of the first PCB. The electrical trances can extend through apertures formed in the first PCB. The second PCB includes electrical traces that extend from the front surface of the second PCB to a back surface of the second PCB. The electrical traces at the back side of the first PCB contact the electrical traces at the front surface of the second PCB. The third PCB includes electrical traces that extend from the front surface of the PCB to another surface of the third PCB, such as a side surface or a back surface. The electrical trances at the back side of the second PCB contact the electrical traces on the front surface of the third PCB. In an implementation, where the electrical traces of the third PCB extend to a side surface of the third PCB, a ribbon cable 1010 with wires 254 is attached to the side of the third PCB where the wires contact the traces. In an implementation, where the electrical traces of the third PCB extend to the back surface of the third PCB, a ribbon cable 1010 with wires 254 is attached to the back surface of the third PCB where the wires contact the traces. FIG. 25 shows an implementation where the ribbon cable attaches to the side of the third PCB. The ribbon cable can be connected to the TIAs via one or more intermediary electrical devices, such as other PCBs or other ribbon cables.

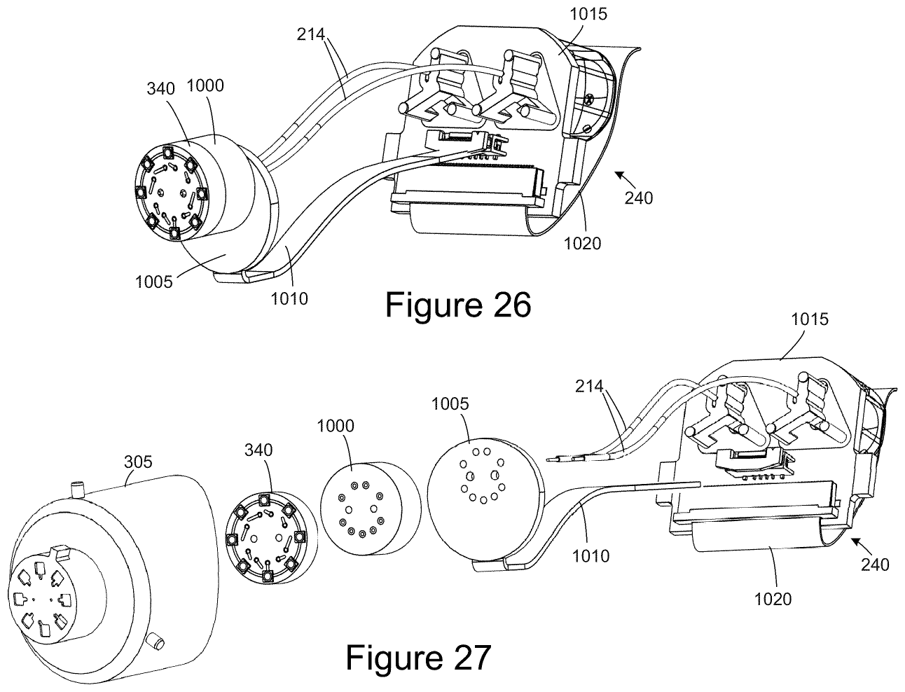

FIGS. 26-27, respectively, show light engine 240 connected to source optical fibers 214 and an exploded view of these elements, in an implementation. One or more PCBs 1015 forming portions of the light engine can be connected to ribbon cable 1010, which in-turn connects to third PCB 1005. One or more PCBs (such as PCB 1015) forming portions of the light engine can be connected to another ribbon cable 1020, which in-turn connects to a PCB that processor 220 may be mounted on. Thereby, the photodetectors are connected to the processor.

FIG. 27 also shows the electrical at the front surface of each of the first, second, and third PCBs, which transfer electrical signals generated by the photodetectors through the light engine to the processor. In an implementation, ribbon cables 1010 and 1020 can be connected to PCB 1015 via edge connectors. Both the second and the third PCBs 1000 and 1005 include apertures that source optical fibers extend through, in an implementation. When the first, second, and third PCBs are connected, the apertures for the optical fibers align so that the source optical fibers can be located in the apertures with little tension on the optical fibers, so little to no light is lost from bending or physical strain on the source optical fibers.

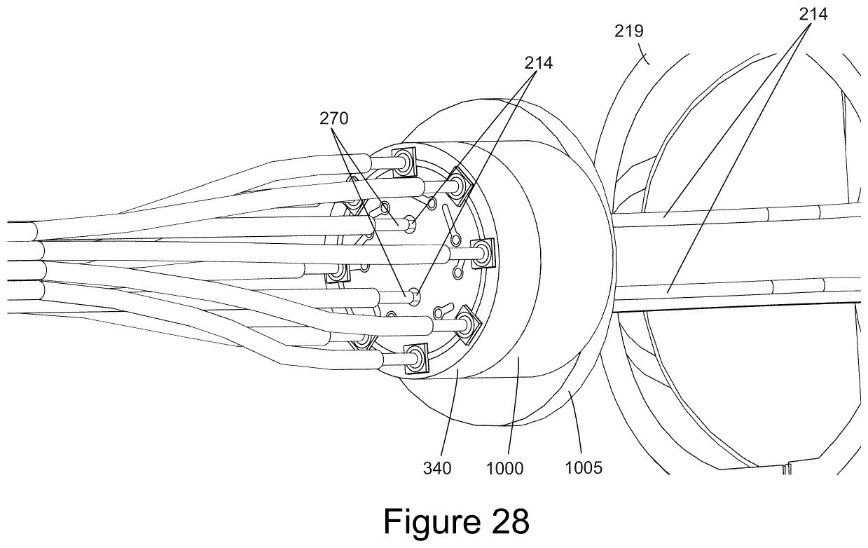

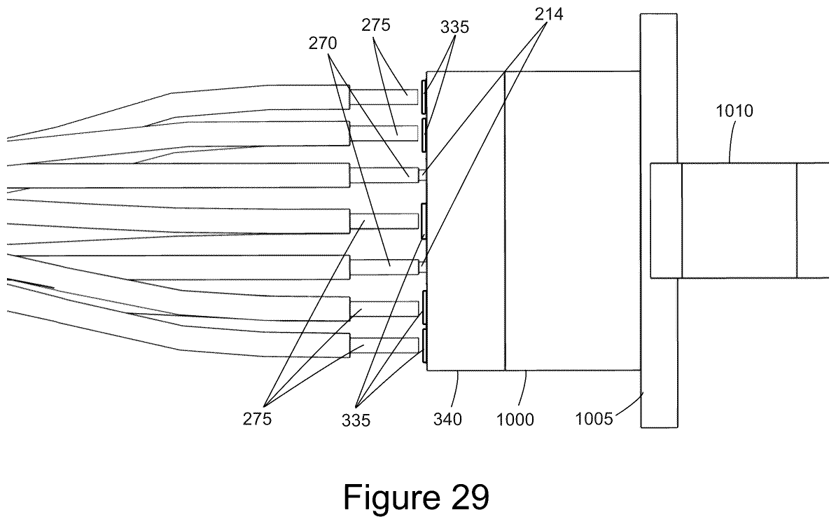

FIGS. 28-29, respectively, show a perspective view and a side vice of source optical fibers 270 tip-to-tip connected to source optical fibers 214. The tips-to-tip connections occurs with when pins 305d of the baffle cap 305 are inserted into the J shaped portions of slots 310b of housing 300. The tips of the source optical fibers can be polished flat to optimize the contact area between the tips of the fibers.

In an implementation, a gap is located between each detector optical fiber 275 and the photodetector 335 that the detector optical fiber is configured to transmit light to. The gap is an air gap, in an implementation. The length of the gap can be from 0.1 millimeters to 5 millimeters. In the implementation where a gap exists between the detector optical fibers and the photodetectors, the tips of the detector optical fibers are inside the apertures formed in the optical coupler where the tips of the detector optical fibers are located. In the implementation, the top surface of the photodetectors can be flush with the top surface of the baffle cap.

In the implementation where a gap exists between the detector optical fibers and the photodetectors, the top surfaces of the photodetectors are inside the apertures of the baffle cap where the top surface of the photodetectors are exposed. In the implementation, the tips of the detector optical fibers can be flush with the top surface of the optical coupler.

In the implementation where a gap exists between the detector optical fibers and the photodetectors, the tips of the detector optical fibers are inside the apertures formed in the optical coupler where the tips of the detector optical fibers are located and the top surfaces of the photodetectors are inside the apertures of the baffle cap where the top surface of the photodetectors are exposed.

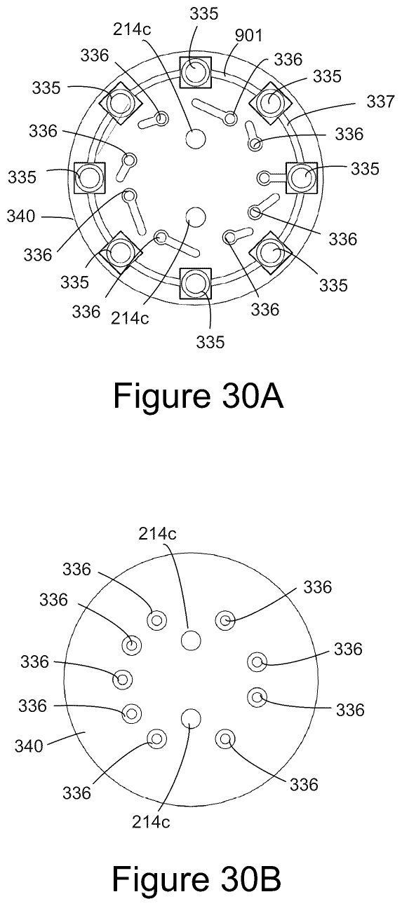

FIGS. 30A-30B, respectively, show front and back views of first PCB 340 on which photodetectors 335 are mounted. In an implementation, the photodetectors are arranged in a circular arrangement on the front surface of the first PCB. The first PCB can include a power plane 901 (e.g., ground or power supply voltage, such as 5 volts), which is configured as a ring where each photodetector is connected to the ring. The first PCB includes a plurality of electrical traces 336 where the electrical traces extend through apertures formed in the first PCB from the front surface to the back surface. The front and back views show the apertures 214c, where the source optical fibers 214 extend through the first PCB.

In an implementation, the elongated tube 315 of the laparoscopic tube embodiments described can be plastic, a plastic-type material, or metal, such as stainless steel or titanium. The housing 219 of the probe unit can be plastic, a plastic-type material, or metal, such as stainless steel or titanium. The laparoscopic tube has a length of 10 centimeters to 100 centimeters. More specifically, the tip of the sensor head can be one of a variety of distances (e.g., 10 centimeters to 100 centimeters) from the top of the laparoscopic tube where the length is based on the particular medical procedure that the laparoscopic oximeter is used for. The outside cross-sectional dimension of the elongated tube of the laparoscopic tube can range from about 3 millimeters to about 20 millimeters or greater (e.g., 3, 4, 5, 6, 7, 8, 9, 10, 15, or 10 millimeters, or others). The cross-sectional shape of the elongated tube of the laparoscopic tube can be, for example, circular, elliptical, oval, or any rounded polygon (e.g., square or rectangle with rounded corners).

The outer face of the sensor head of the laparoscopic tube can have a variety of shapes, such as flat or rounded. In an implementation, the outer face of the sensor head of the laparoscopic tube is not transverse with respect to the lateral cross-section of the elongated tube of the laparoscopic tube. In other embodiments, the outer face of the sensor head is flush with the end of the elongated tube of the laparoscopic tube, recessed within the elongated tube, or extends from the elongated tube. The inner sidewall of elongated tube 315, the outer sidewall of elongated tube 315, or both the inner and outer sidewalls of the laparoscopic tube can also be coated, such as with a polymeric coating to make one or both of the surfaces nonconducting.

FIG. 31 shows laparoscopic oximeter 5 configured to transmit oximetry information to a display 400 via a communication link for the display of the oximetry information on the display. Display 400 may be the display of a surgical tower or a stand-alone display. Laparoscopic oximeter 5 may be configured to communicate with display 400 via a wireless link, a wired link (e.g., cable), or an optical link (e.g., optical fiber).

The communication link can operate according to one of a variety of protocols, such as one of the Bluetooth protocols (e.g., Bluetooth, Bluetooth SMART, Bluetooth Low Energy, others), one of the IEEE 802.11 protocols, ANT, 6LoWPAN, MyriaNed, EnOcean, Z-Wave, Wi-Fi, one of the IEEE 802.15.4 protocol, such as ZigBee, or others. These or other wireless protocols can be used by the laparoscopic oximeter to transfer data from the probe to the display at 1 hertz to 500 kilohertz, such as approximately 250 kilohertz. Data transfer from the display to the laparoscopic oximeter can be at similar data rates.

The wireless link between the laparoscopic oximeter and the display is a direct wireless connection, in an implementation. That is, no intermediary transmitter circuits, receiver circuits, or transceiver circuits receive the wireless signal transmitted from the laparoscopic oximeter for subsequent retransmission of the wireless signal to the display. Similarly, no intermediary transmitter circuits, receiver circuits, or transceiver circuits receive the wireless signal transmitted from the display for subsequent retransmission of the wireless signal to the laparoscopic oximeter.

In an implementation where the laparoscopic oximeter is adapted to communicate with display 400 for the display of information for oxygen saturation measurements, the laparoscopic oximeter does not include a display. In this implementation, display 400 operates as the display for the laparoscopic oximeter. Alternatively, the laparoscopic oximeter may include display 205 and may be adapted to communicate with display 400, where the two displays may display the same, different, or complementary oximetry information.

Display 400 can be a tablet computer or other display type, such as a display that is included in a laparoscopic tower used with other laparoscopic devices used during a laparoscopic surgery. In an implementation where the display is a tablet computer, the display can be attached to a laparoscopic tower that might include other displays and other medical devices. The display can operate an Android mobile operating system or another operating system adapted for use with mobile devices.

Display 400 includes circuitry and a power supply unit that is adapted to supply power to the circuitry. The power supply unit can include circuitry that provides power from a battery of the display. Alternatively, the power supply unit be adapted to receive power from a power outlet (e.g., 120 volts, 220 volts, or other voltages) for powering the display.

The circuitry may be referred to as a processing circuit and can include one or more electrical components or circuits, such as a processor, microprocessor, microcontroller, a multi-core processor, application specific integrated circuit (ASIC), field programmable gate array (FPGA), multiplexers, standard cells, control logic (e.g., programmable logic, programmable logic device (PLD), CPLD, and others), memory, look-up tables, state machines, logic gates, digital signal processors (DSP), and others. In an implementation, the processing circuit performs operations in digital (e.g., Boolean logic). The circuitry can include one or more memories, such as a volatile memory (e.g., a RAM), a nonvolatile memory (e.g., a disk or FLASH), or other memory types.

In an implementation, the circuitry of the display performs the processing of signal data generated by the photodetectors, performs calculations, determines oxygen saturation and other oximeter measurements, and other processing operations to thereafter display oximeter information (e.g., oxygen saturation information) on the display panel.

Display 400 can store and operate one or more computer applications adapted for receiving measurement information for oximeter measurements generated by the laparoscopic oximeter. The display, via the application, can process the information and display the information or a derivative of the information. For example, the laparoscopic oximeter can transmit information (e.g., a value) for blood oxygen saturation (StO2), the percentage of oxygenated hemoglobin (HbO2), the percentage of deoxygenated hemoglobin (Hb), the blood volume, quality metric information for a quality of an oximetry measurement, or other oximetry information. The display can display one or more pieces of information for these values, such as the values themselves or derivatives of the values.

Alternatively, the laparoscopic oximeter can transmit substantially raw measurement data in digital or analog form to the display. Substantially raw measurement data includes data that has not been processed by the processor or any preprocessors. The substantially raw measurement data can be analog detector responses generated by the detectors that may or may not be conditioned, amplified, or both. The substantially raw measurement data can be digitized detector responses that are digitized by the laparoscopic oximeter via the analog-to-digital converter housed within the laparoscopic oximeter. The display, via the use of the application, can perform data processing on the raw measurement data to generate final measurement information for the tissue, such as a value for blood oxygen saturation (StO2), a value for the percentage of oxygenated hemoglobin (HbO2), a value for the percentage of deoxygenated hemoglobin (Hb), blood volume, or other value. The display, via the application, can display one or more pieces of the oximetry information.

Alternatively, the laparoscopic oximeter can transmit partially processed measurement data to the display. Partially processed data can include data for which one or more calibration corrections have been made for intensity differences of light emitted by the LEDs, inherent sensitivity differences of the photodetectors, or other processed information, or any combination of these information. The display, via the use of the application, can perform data processing on the partially processed data to generate final measurement information for the tissue, such as a value for blood oxygen saturation (StO2), a value for the percentage of oxygenated hemoglobin (HbO2), a value for the percentage of deoxygenated hemoglobin (Hb), blood volume, or other value. The display, via the application, can display one or more pieces of the oximetry information.

The battery of the display can include one or more of a variety of battery types, such as one or more disposable batteries or one or more rechargeable batteries. Disposable batteries are discarded after their stored charge is expended. Some disposable battery chemistry technologies include alkaline, zinc carbon, or silver oxide. The battery has sufficient stored charge to allow for use of the handheld device for several hours. In an implementation, the laparoscopic oximeter is a disposable probe.

In implementations where the battery is rechargeable, the battery can be recharged multiple times after the stored charge is expended. Some rechargeable battery chemistry technologies include nickel cadmium (NiCd), nickel metal hydride (NiMH), lithium ion (Li-ion), and zinc air. The battery can be recharged, for example, via an AC adapter with a cord that connects to the handheld unit or via a wireless connection to an induction charger. The circuitry in the display can include a recharger circuit (not shown).

FIG. 32 shows display 400 connected to laparoscopic tube 15 by a detachable cable 1500, in an implementation. Display 400 includes the same or similar circuits and elements as probe unit 10 and connects by detachable cable 1500 to laparoscopic tube 15.

The detachable cable is detachable from display 400 and detachable from laparoscopic tube 15. The detachable cable includes two mechanical couplers with a mechanical coupler at each end of the cable and two optical couplers with an optical coupler at each end of the cable. In an implementation, the detachable cable includes two electrical couplers with an electrical coupler at each end of the cable. The cable includes a number of waveguides, such as optical fibers. The optical fibers can include optical fibers that transmit light from a light engine located in the display to the waveguides in the laparoscopic tube. The optical fibers can include optical fibers that transmit light from the waveguides of the laparoscopic tube to the photodetectors located in the display. In an implementation, the cable includes one or more wires to transmit electrical signals from the display to the laparoscopic tube and from the laparoscopic tube to the display. The cable can include wires in an implementation where the photodetectors are located in the laparoscopic tube and may not include wires where the photodetectors are located in the display.

A first mechanical coupler located at a first end of the detachable cable can apply a first spring force that connects one or more optical fibers of the cable to one or more optical fibers of the display. A second mechanical coupler located at a second end of the cable can apply a second spring force that connects one or more optical fibers of the cable to one or more optical fibers of the laparoscopic tube, such as the one or more optical fibers having ends positioned in the optical coupler of the laparoscopic tube. The mechanical couplers of the detachable cable include one or two twist connectors, one or more a pin connectors, one or more aperture connectors, one or more key structures that guide the alignment of the cable onto the display, laparoscopic tube or both, other connector types, or one or more of these connectors in any combination. The first and second ends of the detachable cable are opposite ends of the cable.

The detachable cable can have a variety of lengths, such as 0.5 meters, 1 meter, 1.5 meters, 2 meters, or longer. The cable can be relatively flexible, allowing the laparoscopic tube to be relatively freely moved for surgery.

With the detachable cable connected to the display and laparoscopic tube, light generated by the light engine is transmitted to the laparoscopic tube through the cable and reflected light that is reflected from tissue is transmitted from the laparoscopic tube to the cable and then through the cable to the display. The light can be detected and converted to electrical signals by the photodetectors located in the display.

The display can process the electrical signals to generate oximetry information, such as oxygen saturation information for tissue being measured. The display can display the oximetry information on the display element. The display element may have a diagonal length across the display of 5-100 centimeters.

Similar to the laparoscopic oximeter embodiments described above, the light engine of the display is electrically connected to the processor and the processor controls the generation of light by the light engine for emission of the light into tissue. The light engine includes one or more sources that generate and transmit light, such as visible light, infrared light, or both. Each source can include one or more light emitting diodes (LEDs), such as one, two, three, four, five, six, seven, eight, nine, ten, or more LEDs. Each LED is adapted to emit one or more wavelengths of light, such as visible light, infrared light, or both. The LEDs can be discrete LEDs, organic LEDs (OLEDs), high brightness LEDs (HLEDs), quantum dot LEDs, laser diodes, or other types of LEDs. One, two, three, four, five, or more LEDs can be optically connected to one optical fiber of the display or the detachable cable.

The photodetectors located in the display can be PIN photodiodes, phototransistors, photoresistors, or other detector type. The display can include one or more TIAs 256 that are connected to the photodetectors. The photodetectors convert received light into electrical signals, which the amplifiers amplify. Each photodetector may be electrically connected to one TIA. In an implementation, multiple photodetectors are connected to a single TIA, for example, via a multiplexer. The TIAs are adapted to receive analog detector responses generated by the photodetectors. The TIAs convert the current for the analog detector responses to a voltage and amplify the voltage.

In some implementations, the display includes one or more electrical components that perform these functions, such as one or more current-to-voltage converters, one or more fixed gain amplifiers, one or more processor-controlled amplifiers, or other amplifiers. It will be understood that a TIA is one example of a current-to-voltage converter that can perform the current-to-voltage conversion and amplification.

The photodetectors may generate output current (e.g., detector responses) that changes relatively linearly with the changing intensity of detected light, whereas the generated output voltage changes relatively nonlinearly with the changing intensity of detected light. The TIA or current-to-voltage converter can output relatively linear voltage from the current-to-voltage conversion. Thereafter, the amplified voltage is also relatively linear.

The display can include one or more analog-to-digital converters. Similar to the probe unit described above, each TIA, fixed gain amplifier, and processor-controlled amplifier may be electrically connected to one ADC. In an implementation, multiple TIAs, fixed gain amplifiers, and processor-controlled amplifiers are connected to one ADC, for example, via a multiplexer. The ADCs are also electrically connected to the processor. The ADCs digitize the amplified voltage signal received from the amplifiers and transfer the digitized detector responses to the processor.

This description of the invention has been presented for the purposes of illustration and description. It is not intended to be exhaustive or to limit the invention to the precise form described, and many modifications and variations are possible in light of the teaching above. Elements of the various implementations can be used with other implementations in a number of ways, such as combinations, substitutions, or both. The implementations were chosen and described in order to best explain the principles of the invention and its practical applications. This description will enable others skilled in the art to best utilize and practice the invention in various implementations and with various modifications as are suited to a particular use. The scope of the invention is defined by the following claims.

The invention claimed is:

1. A method comprising:
forming a housing comprising a first structure and a second structure;
providing the housing comprising the first and second structures is reusable with a plurality of laparoscopic elements that are disposable;
forming a first opening at an end of the first portion;
positioning in the first structure a processor, a memory coupled to the processor, and a light engine coupled to the processor;
forming a second opening at a first end of the second structure;
coupling the second structure to the first structure by coupling the second opening of the second structure to the first opening of the first structure;
forming a first optical interface at a second end of the second structure;
coupling a photodetector to the first optical interface and the processor;
coupling a first optical conductor through the first and second openings to the light engine and the first optical interface;
forming a laparoscopic element to removably couple to the second structure of the housing and comprising a third structure, a fourth structure, and a second optical conductor;
forming a third opening at a first end of the third structure and at a first end of the laparoscopic element;
forming a fourth opening in the third structure oppositely located from the third opening in the third structure;
coupling an interior surface of the third structure over an outside surface of the second structure through the third opening forming a mated connection coupling when the laparoscopic element is removably coupled to the second structure of the housing;
positioning a sensor head at a second end of the fourth structure that is oppositely located from a first end of the fourth structure;
optically coupling the second optical conductor to the first optical conductor in an interior space of the third structure when the laparoscopic element is removably coupled to the second structure of the housing;
coupling the second optical conductor through an interior space of the fourth structure to the sensor head;
optically coupling a third optical conductor to the photodetector in the interior space of the third structure when the laparoscopic element is removably coupled to the second structure of the housing; and
coupling the third optical conductor through the interior space of the fourth structure to the sensor head.

2. The method of claim 1 wherein optically coupling a third optical conductor to the photodetector comprises optically coupling the third optical conductor to the photodetector through an air gap located between the third optical fiber and the photodetector.

3. The method of claim 1 comprising forming a male portion of the second structure;
and forming a female portion of the third coupler, wherein coupling the interior surface of the third structure over the outside surface of the second structure through the third opening forming the mated connection coupling comprises positioning the male portion of the second structure in the female portion of the third coupler.

4. The method of claim 1 comprising providing the third opening of the third structure comprises a first diameter and the fourth opening of the third structure comprises a second diameter less than the first diameter.

5. The method of claim 1 comprising providing the fourth opening is at a position closer to a tip of the laparoscopic element than the first end of the laparoscopic element.

6. The method of claim 1 comprising providing the second optical conductor is longer than the first optical conductor and the third optical conductor is longer than the first optical conductor.

7. The method of claim 1 comprising:
providing the housing comprises a fifth structure;
forming an interior space in the fifth structure;
positioning the second structure at least partially in the interior space of the fifth structure;
forming exterior threads on the fifth structure;
forming exterior threads on the third structure; and
threading the exterior threads into the interior threads forming a first portion of the connection coupling when the housing and laparoscopic element are removably coupled.

8. The method of claim 7 comprising:
providing the laparoscopic element comprises a sixth structure at the first end of the laparoscopic element; and
forming a latched connection between the fifth and sixth structures inside an interior space of the third structure when the housing and laparoscopic element are removably coupled to form a second portion of the connection coupling.

9. The method of claim 7 comprising providing the third structure rotationally couples to the sixth structure.

10. The method of claim 9 comprising:
forming exterior channels on the third structure;
forming interior channels on the fifth structure;
mating the interior channels with the exterior channels forming a rotational interface between the third and fifth structures.

11. The method of claim 1 comprising providing the connection coupling is a spring-loaded connection coupling.

12. The method of claim 1 comprising:
coupling a display to the first portion of the housing and to the processor;
providing the display is visible from an exterior of the first portion.

13. The method of claim 1 comprising positioning the first end of the fourth structure in the interior space through the fourth opening of the third structure.

14. The method of claim 1 comprising positioning the second optical conductor at least partially in an interior space of the fourth structure.

15. The method of claim 1 wherein optically coupling the second optical conductor to the first optical conductor in an interior space of the third structure comprising coupling the first and second optical conductors end-to-end.

16. The method of claim 1 comprising:
positioning a printed circuit board (PCB) in an interior space of the second structure; and
coupling the photodetector to a surface of the PCB.

17. The method of claim 16 comprising facing the surface of the PCB on which the photodetector is located towards the sensor head of the laparoscopic element.

18. The method of claim 1 comprising:
providing the second structure comprises a first axis extending through the first opening of the first structure and the first optical interface positioned opposite of the first opening of the first structure;
providing the third structure comprises a second axis extending through the third opening of the third structure and the fourth opening of the third structure;
providing the fourth structure comprises a third axis extending through the sensor head and an opening at the first end of the fourth structure; and
axially aligning the first, second, and third axes when the housing and laparoscopic element are removably coupled.

19. A method comprising:
forming a housing comprising a first structure and a second structure;
providing the housing comprising the first and second structures is reusable with a plurality of laparoscopic elements that are disposable;
forming a first opening at an end of the first portion;
positioning in the first structure a processor, a memory coupled to the processor, and a light engine coupled to the processor;
forming a second opening at a first end of the second structure;
coupling the second structure to the first structure by coupling the second opening of the second structure to the first opening of the first structure;
forming a first optical interface at a second end of the second structure;
coupling a photodetector to a printed circuit board (PCB) in an interior space of the second structure;
coupling the photodetector to the processor via the PCB;
coupling a first optical conductor through the first and second openings to the light engine and the first optical interface;
forming a laparoscopic element to removably couple to the second structure of the housing and comprising a third structure, a fourth structure, and a second optical conductor;
forming a third opening at a first end of the third structure and at a first end of the laparoscopic element;
forming a fourth opening in the third structure oppositely located from the third opening in the third structure;
coupling an interior surface of the third structure over an outside surface of the second structure through the third opening forming a mated connection coupling when the laparoscopic element is removably coupled to the second structure of the housing;
positioning a sensor head at a second end of the fourth structure that is oppositely located from a first end of the fourth structure;
optically coupling the second optical conductor to the first optical conductor in an interior space of the third structure when the laparoscopic element is removably coupled to the second structure of the housing;
coupling the second optical conductor through an interior space of the fourth structure to the sensor head;
optically coupling a third optical conductor to the photodetector in the interior space of the third structure when the laparoscopic element is removably coupled to the second structure of the housing; and
coupling the third optical conductor through the interior space of the fourth structure to the sensor head.

20. The method of claim 19 comprising facing the surface of the PCB on which the photodetector is located towards the sensor head of the laparoscopic element.

21. The method of claim 19 comprising;
providing the second structure comprises a first axis extending through the first opening of the first structure and the first optical interface positioned opposite of the first opening of the first structure;
providing the third structure comprises a second axis extending through the third opening of the third structure and the fourth opening of the third structure;
providing the fourth structure comprises a third axis extending through the sensor head and an opening at the first end of the fourth structure; and
axially aligning the first, second, and third axes when the housing and laparoscopic element are removably coupled.

22. The method of claim 21 comprising:
providing the laparoscopic element comprises a fifth structure located in an interior space of the third structure;
forming a first key structure on the fifth structure;
forming a second key structure on the second structure;
mating the first and second key structures when the housing and laparoscopic element are removably coupled; and
wherein axially aligning the first, second, and third axes when the housing and laparoscopic element are removably coupled comprises axially aligning the first, second, and third axes when the first and second key structures are mated.

23. The method of claim 19 comprising:
coupling a fourth optical conductor through the first and second openings to the light engine and the first optical interface;
optically coupling a fifth optical conductor to the fourth optical conductor in an interior space of the third structure when the laparoscopic element is removably coupled to the second structure of the housing; and
coupling the fifth optical conductor through an interior space of the fourth structure to the sensor head.

24. The method of claim 23 comprising:
providing the second optical fiber is longer than the first optical fiber;
the third optical fiber is longer than the first optical fiber and is longer than the fourth optical fiber; and
the fifth optical fiber is longer than the first optical fiber and is longer than the fourth optical fiber.

25. The method of claim 19 wherein optically coupling the third optical conductor to the photodetector in the interior space of the third structure comprises optically coupling the third optical conductor to the photodetector through an air gap.

26. The method of claim 19 wherein optically coupling the second optical conductor to the first optical conductor in an interior space of the third structure comprises physically coupling the first and second optical conductors end-to-end.

27. The method of claim 19 comprising:
forming a first numerical aperture of a first core of the first optical fiber; and
forming a second numerical aperture of a second core of the second optical fiber that is greater than the first numerical aperture.
